(19) United States
(12) Patent Application Publication
(10) Pub. No.: US 2016/0175379 A1
(43) Pub. Date: Jun. 23, 2016

(54) COMBINATION THERAPY FOR THE TREATMENT OF ISCHEMIA-REPERFUSION INJURY

(71) Applicant: STEALTH BIO THERAPEUTICS CORP, Monte-Carlo (MC)

(72) Inventor: D. Travis Wilson, Newton, MA (US)

(73) Assignee: STEALTH BIO THERAPEUTICS CORP, Monte-Carlo (MC)

(21) Appl. No.: 14/910,440

(22) PCT Filed: Aug. 12, 2014
(86) PCT No.: PCT/US14/50747

§ 371 Date: Feb. 5, 2016

Related U.S. Application Data
(60) Provisional application No. 61/864,843, filed on Aug. 12, 2013.

(51) Int. Cl. A61K 38/06; A61K 45/06

(52) U.S. Cl. CPC A61K 38/06; A61K 45/06

(57) ABSTRACT

The present technology provides methods of preventing or treating an ischemia-reperfusion injury, such as acute myocardial infarction injury, in a mammalian subject. The methods comprise administering to the subject an effective amount of an aromatic-cationic peptide and a second active agent to subjects in need thereof.

### CROSS-REFERENCE TO RELATED APPLICATIONS

This application claims the benefit of and priority to U.S. Provisional Application No. 61/864,843, filed Aug. 12, 2013, the content of which is incorporated herein by reference in its entirety.

### TECHNICAL FIELD

The present technology relates generally to compositions and methods of preventing or treating ischemia-reperfusion injury. In particular, embodiments of the present technology relate to administering aromatic-cationic peptides and an agent in effective amounts to prevent or treat ischemia reperfusion injury, such as acute myocardial infarction injury, in mammalian subjects.

### SUMMARY

The present technology relates to the treatment or prevention of ischemia-reperfusion injury in mammals through the administration of a therapeutically effective amount of aromatic-cationic peptides and a second active agent. The present technology also relates to the treatment or prevention of acute myocardial infarction (AMI) injury in mammals through administration of therapeutically effective amounts of aromatic-cationic peptides and one or more cardiovascular agents to subjects in need thereof.

In one aspect, the present disclosure relates to the use of an aromatic-cationic peptide and a cardiovascular agent in the manufacture of a medicament for reducing infarct size and apoptotic cell death produced by AMI, wherein the peptide is D-Arg-2′6′-Dmt-Lys-Phe-NH₂; the cardiovascular agent is one or more of hyaluronidase, a corticosteroid, recombinant superoxide dismutase, prostacyclin, fluosol, magnesium, poloxamer 188, trimetazidine, eniporidine, cariporidine, a nitrate, anti-P selectin, an anti-CD18 antibody, adenosine, and glucose-insulin-potassium; and wherein the medicament reduces infarct size and apoptotic cell death by at least 50% as compared to an untreated control.

In some embodiments, the cardiovascular agent is one or more of recombinant superoxide dismutase, magnesium, a nitrate, anti-P selectin, an anti-CD18 antibody, adenosine, and glucose-insulin-potassium.

In other embodiments, the cardiovascular agent is one or more of hyaluronidase, prostacyclin, fluosol, poloxamer 188, trimetazidine, eniporidine, and cariporidine.

In certain embodiments, the cardiovascular agent is one or more corticosteroids selected from the group consisting of hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate.

In another aspect, the present disclosure relates to the use of an aromatic-cationic peptide and a cardiovascular agent in the manufacture of a medicament for reducing infarct size and apoptotic cell death produced by AMI, wherein the peptide is D-Arg-2′6′-Dmt-Lys-Phe-NH₂; the cardiovascular agent is one or more of an anti-arrhythmia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a thromboxane receptor antagonist, a radical scavenger, an anti-platelet drug, a β-adrenaline receptor blocking drug, an α-receptor blocking drug, a sympathetic nerve inhibitor, a digitalis formulation, an inotrope, and an antihyperlipidemic drug; and wherein the medicament reduces infarct size and apoptotic cell death by at least 50% as compared to an untreated control.

In some embodiments, the cardiovascular agent is one or more anti-arrhythmia agents selected from the group consisting of lidocaine, lignocaine moricizine, mexiletine, tocainide, procainamide, encainide, flecanide, tocainide, phenytoin, propafenone, quinidine, disopyramide, flecainide, propranolol, esmolol, amiodarone, artilide, bretylium, clofilium, isobutilide, sotalol, azimilide, dofetilide, dronedarone, ersentilide, ibutilide, tedisamil, trecetilide, verapamil, diltaizem, digitalis, adenosine, nickel chloride, and magnesium ions.

In some embodiments, the cardiovascular agent is one or more vasodilators selected from the group consisting of bencyclane, cinnarizine, citicoline, cyclandelate, cyclonicate, ebumamonine, hydralazine phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostaglandin E1, prostaglandin I2, an endothelin receptor blocking drug, diltiazem, nicorandil, and nitroglycerin.

In some embodiments, the cardiovascular agent is one or more anti-anginal agents selected from the group consisting of nitrates, isosorbide nitrate, glyceryl trinitrate, and pentaerythritol tetranitrate.

In some embodiments, the cardiovascular agent is one or more cardioglycosides selected from the group consisting of digoxin and digitoxin.

In certain embodiments, the cardiovascular agent is one or more diuretics selected from the group consisting of thiazide diuretics, loop diuretics, K+ sparing diuretics, osmotic diuretics, nonthiazide diuretics, and acetazolamide.

In other embodiments, the cardiovascular agent is one or more sedatives selected from the group consisting of nitrazepam, flurazepam and diazepam.

In some embodiments, the cardiovascular agent is one or more ACE inhibitors selected from the group consisting of captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril.

In certain embodiments, the cardiovascular agent is one or more angiotensin II antagonists selected from the group consisting of losartan, candesartan, valsartan, eprosartan, and irbesartan.

In some embodiments, the cardiovascular agent is one or more thrombolytic agents selected from the group consisting of tissue-type plasminogen activators, nasaruplase, streptokinase, urokinase, prourokinase, anisoylated plasminogen streptokinase activator complex, aspirin, heparin, warfarin that inhibits Vit K-dependent factors, low molecular weight heparins that inhibit factors X and II, thrombin inhibitors, inhibitors of platelet GP IIbIIIa receptors, inhibitors of tissue factor (TF), inhibitors of human von Willebrand factor, reptilase, TNK-t-PA, staphylokinase, and animal salivary gland plasminogen activators.

In some embodiments, the cardiovascular agent is one or more calcium channel blockers selected from the group consisting of aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline.

In certain embodiments, the cardiovascular agent is one or more thromboxane receptor antagonists selected from the group consisting of ifetroban, prostacyclin mimetics, and phosphodiesterase inhibitors.

In some embodiments, the cardiovascular agent is one or more antiplatelet drugs selected from the group consisting of ticlopidine hydrochloride, dipyridamole, cilostazol, ethyl icosapentate, sarpogrelate hydrochloride, dilazep hydrochloride, trapidil, a nonsteroidal antiinflammatory agent, beraprostsodium, iloprost, and indobufene.

In other embodiments, the cardiovascular agent is one or more β-adrenaline receptor blocking drugs selected from the group consisting of propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol.

In some embodiments, the cardiovascular agent is one or more α-receptor blocking drugs selected from the group consisting of amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine.

In certain embodiments, the cardiovascular agent is one or more sympathetic nerve inhibitors selected from the group consisting of clonidine, guanfacine, guanabenz, methyldopa, reserpine, hydralazine, todralazine, budralazine, and cadralazine.

In some embodiments, the cardiovascular agent is one or more digitalis formulations selected from the group consisting of digitoxin, digoxin, methyldigoxin, deslanoside, vesnarinone, lanatoside C, and proscillaridin.

In one aspect, the present disclosure provides a pharmaceutical composition comprising (i) a peptide D-Arg-2′6′-Dmt-Lys-Phe-NH₂ or a pharmaceutically acceptable salt, such as acetate or trifluoroacetate salt, and (ii) a second active agent, e.g. a cardiovascular agent.

In another aspect, the present disclosure provides a kit for treating an acute myocardial infarction injury in a mammalian subject comprising: (i) a peptide D-Arg-2′6′-Dmt-Lys-Phe-NH₂ or a pharmaceutically acceptable salt such as acetate or trifluoroacetate salt, and (ii) a cardiovascular agent, wherein the peptide and cardiovascular agent are packaged in the same or separate vials.

In another aspect, the present disclosure provides a kit for treating ischemia-reperfusion injury in a mammalian subject comprising: (i) a peptide D-Arg-2′6′-Dmt-Lys-Phe-NH₂ or a pharmaceutically acceptable salt such as acetate or trifluoroacetate salt, and (ii) a second active agent, wherein the peptide and active agent are packaged in the same or separate vials.

In one aspect, the present disclosure provides a method for treating an acute myocardial infarction injury in a mammalian subject, the method comprising administering simultaneously, separately or sequentially an effective amount of (i) a peptide D-Arg-2′6′-Dmt-Lys-Phe-NH₂ or a pharmaceutically acceptable salt, such as acetate or trifluoroacetate salt, and (ii) a cardiovascular agent.

In one embodiment, the cardiovascular agent is selected from the group consisting of: hyaluronidase, a corticosteroid, recombinant superoxide dismutase, prostacyclin, fluosol, magnesium, poloxamer 188, trimetazidine, eniporidine, cariporidine, a nitrate, anti-P selectin, an anti-CD18 antibody, adenosine, and glucose-insulin-potassium. In one embodiment, the cardiovascular agent is selected from the group consisting of: an anti-arrhythmia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a thromboxane receptor antagonist, a radical scavenger, an anti-platelet drug, a β-adrenaline receptor blocking drug, α-receptor blocking drug, a sympathetic nerve inhibitor, a digitalis formulation, an inotrope, and an antihyperlipidemic drug. In one embodiment, the cardiovascular agent is cyclosporine.

In one embodiment, the peptide and the active agent are administered sequentially in either order. In one embodiment, the peptide and the active agent are administered sequentially in either order prior to performing a revascularization procedure on the subject. In one embodiment, the peptide and the active agent are administered simultaneously.

In one embodiment, the peptide and the cardiovascular agent are administered simultaneously prior to performing a revascularization procedure on the subject. In one embodiment, the subject is administered the peptide and the cardiovascular agent after a revascularization procedure. In one embodiment, the subject is administered the peptide and the cardiovascular agent simultaneously or separately during and after performing a revascularization procedure on the subject. In one embodiment, the subject is administered the peptide continuously before, during, and after a revascularization procedure and the subject is administered the cardiovascular agent as a bolus dose immediately prior to the revascularization procedure. In one embodiment, the subject is administered the cardiovascular agent before a revascularization procedure and the subject is administered the peptide continuously during and after the revascularization procedure. In one embodiment, the subject is administered the cardiovascular agent continuously before and during a revascularization procedure and the subject is administered the peptide continuously during and after the revascularization procedure.

In one embodiment, the subject is administered the peptide for at least 3 hours after the revascularization procedure. In one embodiment, the subject is administered the peptide for at least 5 hours after the revascularization procedure. In one embodiment, the subject is administered the peptide for at least 8 hours after the revascularization procedure. In one embodiment, the subject is administered the peptide for at least 12 hours after the revascularization procedure. In one embodiment, the subject is administered the peptide for at least 24 hours after the revascularization procedure.

In one embodiment, the subject is administered the peptide starting at least 8 hours before the revascularization procedure. In one embodiment, the subject is administered the peptide starting at least 4 hours before the revascularization procedure. In one embodiment, the subject is administered the peptide starting at least 2 hours before the revascularization procedure. In one embodiment, the subject is administered the peptide starting at least 1 hour before the revascularization procedure. In one embodiment, the subject is administered the peptide starting at least 30 minutes before the revascularization procedure.

In one embodiment, the revascularization procedure is selected from the group consisting of: percutaneous coronary intervention; balloon angioplasty; insertion of a bypass graft; insertion of a stent; or directional coronary atherectomy. In one embodiment, the revascularization procedure is removal of the occlusion.

In another aspect, the present disclosure provides a method of coronary revascularization comprising: (a) administering simultaneously, separately or sequentially an effective amount of (i) a peptide D-Arg-2′6′-Dmt-Lys-Phe-NH₂ or a pharmaceutically acceptable salt such as acetate or trifluoroacetate salt, and (ii) a cardiovascular agent; and (b) performing a coronary artery bypass graft procedure on the subject.

In some embodiments, the aromatic-cationic peptide is a peptide having:

at least one net positive charge;

a minimum of four amino acids;

a maximum of about twenty amino acids;

a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1. In particular embodiments, the mammalian subject is a human.

In one embodiment, 2pₘ is the largest number that is less than or equal to r+1, and a may be equal to pₜ. The aromatic-cationic peptide may be a water-soluble peptide having a minimum of two or a minimum of three positive charges.

In one embodiment, the peptide comprises one or more non-naturally occurring amino acids, for example, one or more D-amino acids. In some embodiments, the C-terminal carboxyl group of the amino acid at the C-terminus is amidated. In certain embodiments, the peptide has a minimum of four amino acids. The peptide may have a maximum of about 6, a maximum of about 9, or a maximum of about 12 amino acids.

In one embodiment, the peptide comprises a tyrosine or a 2′,6′-dimethyltyrosine (Dmt) residue at the N-terminus. For example, the peptide may have the formula Tyr-D-Arg-Phe-Lys-NH₂ or 2′,6′-Dmt-D-Arg-Phe-Lys-NH₂. In another embodiment, the peptide comprises a phenylalanine or a 2′,6′-dimethylphenylalanine residue at the N-terminus. For example, the peptide may have the formula Phe-D-Arg-Phe-Lys-NH₂ or 2′,6′-Dmp-D-Arg-Phe-Lys-NH₂. In a particular embodiment, the aromatic-cationic peptide has the formula D-Arg-2′,6′-Dmt-Lys-Phe-NH₂.

In one embodiment, the peptide is defined by formula I:

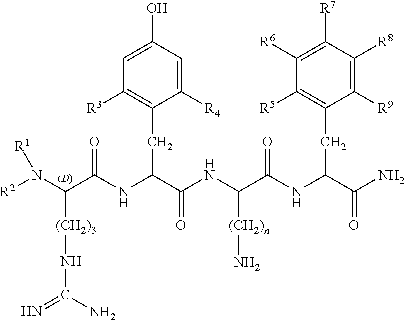

wherein R¹ and R² are each independently selected from

(i) hydrogen;

(ii) linear or branched C₁-C₆ alkyl;

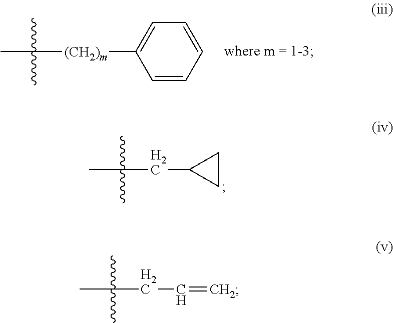

R³ and R⁴ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo;

R⁵, R⁶, R⁷, R⁸, and R⁹ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and n is an integer from 1 to 5.

In a particular embodiment, R¹ and R² are hydrogen; R³ and R⁴ are methyl; R⁵, R⁶, R⁷, R⁸, and R⁹ are all hydrogen; and n is 4.

In one embodiment, the peptide is defined by formula II:

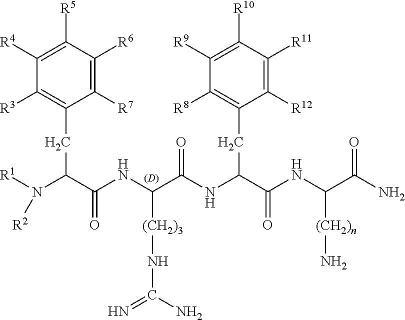

wherein R¹ and R² are each independently selected from

(i) hydrogen;

(ii) linear or branched C₁-C₆ alkyl;

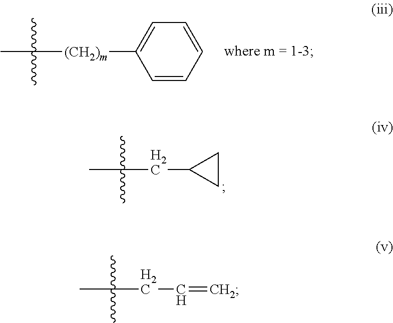

R³, R⁴, R⁵, R⁶, R⁷, R⁸, R⁹, R¹⁰, R¹¹ and R¹² are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and n is an integer from 1 to 5.

In a particular embodiment, R¹, R², R³, R⁴, R⁵, R⁶, R⁷, R⁸, R⁹, R¹⁰, R¹¹, and R¹² are all hydrogen; and n is 4. In another embodiment, R¹, R², R³, R⁴, R⁵, R⁶, R⁷, R⁸, R⁹, and R¹¹ are all hydrogen; R⁸ and R¹² are methyl; R¹⁰ is hydroxyl; and n is 4.

The aromatic-cationic peptides may be administered in a variety of ways. In some embodiments, the peptides may be administered orally, topically, intranasally, intraperitoneally, intravenously, subcutaneously, or transdermally (e.g., by iontophoresis).

### DETAILED DESCRIPTION

It is to be appreciated that certain aspects, modes, embodiments, variations and features of the present technology are described below in various levels of detail in order to provide a substantial understanding of the present technology.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N. Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the terms “cardiovascular agent” or “cardiovascular drug” refers to a therapeutic compound that is useful for treating or preventing a cardiovascular disease or condition. Non-limiting examples of suitable cardiovascular agents include ACE inhibitors (angiotensin II converting enzyme inhibitors), ARB's (angiotensin II receptor antagonists), adrenergic blockers, adrenergic agonists, anti-anginal agents, anti-arrhythmics, anti-platelet agents, anti-coagulants, anti-hypertensives, anti-lipemic agents, calcium channel blockers, COX-2 inhibitors, diuretics, endothelin receptor antagonists, HMG Co-A reductase inhibitors, inotropic agents, rennin inhibitors, vasodilators, vasopressors, AGE crosslink breakers, and AGE formation inhibitors (advanced glycosylation end-product formation inhibitors, such as pimagedine), and combinations thereof.

As used herein, the term “effective amount” refers to a quantity sufficient to achieve a desired therapeutic and/or prophylactic effect, e.g., an amount which results in the prevention of, or a decrease in, cardiac ischemia-reperfusion injury or one or more symptoms associated with cardiac ischemia-reperfusion injury. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In the methods described herein, the aromatic-cationic peptides and cardiovascular agent may be administered to a subject having one or more signs or symptoms of acute myocardial infarction injury. In other embodiments, the mammal has one or more signs or symptoms of myocardial infarction, such as chest pain described as a pressure sensation, fullness, or squeezing in the mid portion of the thorax; radiation of chest pain into the jaw or teeth, shoulder, arm, and/or back; dyspnea or shortness of breath; epigastric discomfort with or without nausea and vomiting; and diaphoresis or sweating. For example, a “therapeutically effective amount” of the aromatic-cationic peptides and/or cardiovascular agent is meant levels in which the physiological effects of an acute myocardial infarction injury are, at a minimum, ameliorated.

As used herein the term “ischemia reperfusion injury” refers to the damage caused first by hypoxia in a tissue followed by the sudden perfusion of oxygen to the deprived tissue. In one embodiment, the hypoxia is caused first by restriction of the blood supply to a tissue and the reperfusion is due to a sudden resupply of blood.

An “isolated” or “purified” polypeptide or peptide is substantially free of cellular material or other contaminating polypeptides from the cell or tissue source from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include enzymes, hormones and other proteinaceous and nonproteinaceous solutes.

As used herein, the terms “polypeptide”, “peptide”, and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the terms “treating” or “treatment” or “alleviation” refers to therapeutic measures, wherein the object is to ameliorate or slow down (lessen) the progression of the targeted pathologic condition or disorder. A subject is successfully “treated” for ischemia reperfusion injury if, after receiving a therapeutic amount of the aromatic-cationic peptides and cardiovascular agent according to the methods described herein, the subject shows observable and/or measurable reduction in or absence of one or more signs and symptoms of ischemia reperfusion injury, such as, e.g., reduced infarct size. It is also to be appreciated that the various modes of treatment or prevention of medical conditions as described are intended to mean “substantial”, which includes total but also less than total treatment or prevention, and wherein some biologically or medically relevant result is achieved.

As used herein, “prevention” or “preventing” of a disorder or condition refers to one or more compounds that, in a statistical sample, reduces the occurrence of the disorder or condition in the treated sample relative to an untreated control sample, or delays the onset of the disorder or condition relative to the untreated control sample. As used herein, preventing ischemia-reperfusion injury includes preventing oxidative damage or preventing mitochondrial permeability transitioning, thereby preventing or ameliorating the harmful effects of the loss and subsequent restoration of blood flow to the heart.

### Methods of Prevention or Treatment

The present technology relates to the treatment or prevention of ischemia-reperfusion injury by administration of certain aromatic-cationic peptides and one or more active agents to a subject in need thereof. The present technology also relates to the treatment or prevention of acute myocardial infarction injury by administration of certain aromatic-cationic peptides and one or more cardiovascular agents to a subject in need thereof. In some embodiments, the therapeutic agents are administered in conjunction with a revascularization procedure. Also provided is a method for the treatment or prevention of cardiac ischemia-reperfusion injury. Also provided is a method of treating a myocardial infarction in a subject to prevent injury to the heart upon reperfusion. In one aspect, the present technology relates to a method of coronary revascularization comprising administering to a mammalian subject a therapeutically effective amount of the aromatic cationic peptide and one or more cardiovascular agents and performing coronary artery bypass graft (CABG) procedure on the subject.

In one embodiment, the aromatic-cationic peptides and/or one or more agents are administered in dosages that are sub-therapeutic for each agent when administered separately. However, the combination of the two agents results in synergism, which provides an enhanced effect that is not observed when each of the agents are administered individually at higher doses. In one embodiment, the administration of the aromatic-cationic peptide and one or more agents “primes” the tissue, so that it is more responsive to the therapeutic effects of the other agent. For this reason, a lower dose of the aromatic-cationic peptide and one or more agents can be administered, and yet, a therapeutic effect is still observed.

In one embodiment, the subject is administered the peptide and one or more cardiovascular agents simultaneously, separately, or sequentially prior to a revascularization procedure. In another embodiment, the subject is administered the peptide and one or more cardiovascular agents simultaneously, separately, or sequentially after the revascularization procedure. In another embodiment, the subject is administered the peptide and one or more cardiovascular agents simultaneously, separately, or sequentially during and after the revascularization procedure. In yet another embodiment, the subject is administered the peptide and one or more cardiovascular agents simultaneously or separately continuously before, during, and after the revascularization procedure. In another embodiment, the subject is administered the peptide and one or more cardiovascular agents regularly (i.e., chronically) following an AMI and/or a revascularization or CABG procedure.

In one embodiment, the subject is administered the peptide and/or one or more cardiovascular agents for at least 3 hours, at least 5 hours, at least 8 hours, at least 12 hours, or at least 24 hours after the revascularization procedure. In one embodiment, the subject is administered the peptide and/or one or more cardiovascular agents starting at least 8 hours, at least 4 hours, at least 2 hours, at least 1 hour, or at least 30 minutes prior to the revascularization procedure. In one embodiment, the subject is administered the peptide and/or one or more cardiovascular agents for at least one week, at least one month or at least one year after the revascularization procedure.

Aromatic-cationic peptides are water-soluble and highly polar. Despite these properties, the peptides can readily penetrate cell membranes. The aromatic-cationic peptides typically include a minimum of three amino acids or a minimum of four amino acids, covalently joined by peptide bonds. The maximum number of amino acids present in the aromatic-cationic peptides is about twenty amino acids covalently joined by peptide bonds. Suitably, the maximum number of amino acids is about twelve, more preferably about nine, and most preferably about six.

The amino acids of the aromatic-cationic peptides can be any amino acid. As used herein, the term “amino acid” is used to refer to any organic molecule that contains at least one amino group and at least one carboxyl group. Typically, at least one amino group is at the a position relative to a carboxyl group. The amino acids may be naturally occurring. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Other naturally occurring amino acids include, for example, amino acids that are synthesized in metabolic processes not associated with protein synthesis. For example, the amino acids ornithine and citrulline are synthesized in mammalian metabolism during the production of urea. Another example of a naturally occurring amino acid includes hydroxyproline (Hyp).

The peptides optionally contain one or more non-naturally occurring amino acids. For example, the peptide may have no amino acids that are naturally occurring. The non-naturally occurring amino acids may be levorotary (L-), dextrorotatory (D-), or mixtures thereof. Non-naturally occurring amino acids are those amino acids that typically are not synthesized in normal metabolic processes in living organisms, and do not naturally occur in proteins. In addition, the non-naturally occurring amino acids suitably are also not recognized by common proteases. The non-naturally occurring amino acid can be present at any position in the peptide. For example, the non-naturally occurring amino acid can be at the N-terminus, the C-terminus, or at any position between the N-terminus and the C-terminus.

The non-natural amino acids may, for example, comprise alkyl, aryl, or alkylaryl groups not found in natural amino acids. Some examples of non-natural alkyl amino acids include α-aminobutyric acid, β-aminobutyric acid, γ-aminobutyric acid, δ-aminovaleric acid, and ε-aminocaproic acid. Some examples of non-natural aryl amino acids include ortho-, meta, and para-aminobenzoic acid. Some examples of non-natural alkylaryl amino acids include ortho-, meta-, and para-aminophenylacetic acid, and γ-phenyl-β-aminobutyric acid. Non-naturally occurring amino acids include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva) and norleucine (Nle).

Another example of a modification of an amino acid in a peptide is the derivatization of a carboxyl group of an aspartic acid or a glutamic acid residue of the peptide. One example of derivatization is amidation with ammonia or with a primary or secondary amine, e.g. methylamine, ethylamine, dimethylamine or diethylamine. Another example of derivatization includes esterification with, for example, methyl or ethyl alcohol. Another such modification includes derivatization of an amino group of a lysine, arginine, or histidine residue. For example, such amino groups can be acylated. Some suitable acyl groups include, for example, a benzoyl group or an alkanoyl group comprising any of the C₁-C₄ alkyl groups mentioned above, such as an acetyl or propionyl group.

The non-naturally occurring amino acids are preferably resistant, and more preferably insensitive, to common proteases. Examples of non-naturally occurring amino acids that are resistant or insensitive to proteases include the dextrorotatory (D-) form of any of the above-mentioned naturally occurring L-amino acids, as well as L- and/or D-non-naturally occurring amino acids. The D-amino acids do not normally occur in proteins, although they are found in certain peptide antibiotics that are synthesized by means other than the normal ribosomal protein synthetic machinery of the cell. As used herein, the D-amino acids are considered to be non-naturally occurring amino acids.

In order to minimize protease sensitivity, the peptides should have less than five, preferably less than four, more preferably less than three, and most preferably, less than two contiguous L-amino acids recognized by common proteases, irrespective of whether the amino acids are naturally or non-naturally occurring. Optimally, the peptide has only D-amino acids, and no L-amino acids. If the peptide contains protease sensitive sequences of amino acids, at least one of the amino acids is preferably a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine.

The aromatic-cationic peptides should have a minimum number of net positive charges at physiological pH in comparison to the total number of amino acid residues in the peptide. The minimum number of net positive charges at physiological pH will be referred to below as (pₘ). The total number of amino acid residues in the peptide will be referred to below as (r). The minimum number of net positive charges discussed below are all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

“Net charge” as used herein refers to the balance of the number of positive charges and the number of negative charges carried by the amino acids present in the peptide. In this specification, it is understood that net charges are measured at physiological pH. The naturally occurring amino acids that are positively charged at physiological pH include L-lysine, L-arginine, and L-histidine. The naturally occurring amino acids that are negatively charged at physiological pH include L-aspartic acid and L-glutamic acid. Typically, a peptide has a positively charged N-terminal amino group and a negatively charged C-terminal carboxyl group. The charges cancel each other out at physiological pH.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≦ p + 1)

| | (r) | | | | | | | | | | | | | | | | | |
|---|---|---|---|---|---|---|---|---|---|---|---|---|---|---|---|---|---|---|
| | 3 | 4 | 5 | 6 | 7 | 8 | 9 | 10 | 11 | 12 | 13 | 14 | 15 | 16 | 17 | 18 | 19 | 20 |
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≦ p + 1)

| | (r) | | | | | | | | | | | | | | | | | |
|---|---|---|---|---|---|---|---|---|---|---|---|---|---|---|---|---|---|---|
| | 3 | 4 | 5 | 6 | 7 | 8 | 9 | 10 | 11 | 12 | 13 | 14 | 15 | 16 | 17 | 18 | 19 | 20 |
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, suitably, a minimum of two net positive charges and more preferably a minimum of three net positive charges.

It is also important that the aromatic-cationic peptides have a minimum number of aromatic groups in comparison to the total number of net positive charges (pₜ). The minimum number of aromatic groups will be referred to below as (a). Naturally occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≦ pₜ + 1 or a = pₜ = 1)

| | (pₜ) | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≦ pₜ + 1 or a = pₜ = 1)

| | (pₜ) | | | | | | | | | | | | | | | | | | | |
|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pₜ) are equal. In one embodiment, the aromatic-cationic peptide is a tripeptide having two net positive charges and at least one aromatic amino acid. In a particular embodiment, the aromatic-cationic peptide is a tripeptide having two net positive charges and two aromatic amino acids.

Carboxyl groups, especially the terminal carboxyl group of a C-terminal amino acid, are suitably amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethylamido, N-methyl-N-ethylamido, N-phenylamido or N-phenyl-N-ethylamido group. The free carboxylate groups of the asparagine, glutamine, aspartic acid, and glutamic acid residues not occurring at the C-terminus of the aromatic-cationic peptides may also be amidated wherever they occur within the peptide. The amidation at these internal positions may be with ammonia or any of the primary or secondary amines described above.

Aromatic-cationic peptides include, but are not limited to, the following peptide examples:

Lys-D-Arg-Tyr-NH₂
Phe-D-Arg-His
D-Tyr-Trp-Lys-NH₂
Trp-D-Lys-Tyr-Arg-NH₂
Tyr-His-D-Gly-Met
Phe-Arg-D-His-Asp
Tyr-D-Arg-Phe-Lys-Glu-NH₂
Met-Tyr-D-Lys-Phe-Arg
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-Tyr-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-His-D-Lys-Arg-
Trp-NH₂
Ala-D-Phe-D-Arg-Tyr-Lys-D-Trp-His-D-Tyr-Gly-
Phe
Tyr-D-His-Phe-D-Arg-Asp-Lys-D-Arg-His-Trp-D-
His-Phe
Phe-Phe-D-Tyr-Arg-Glu-Asp-D-Lys-Arg-D-Arg-His-
Phe-NH₂
Phe-Try-Lys-D-Arg-Trp-His-D-Lys-D-Lys-Glu-Arg-
D-Tyr-Thr
Tyr-Asp-D-Lys-Tyr-Phe-D-Lys-D-Arg-Phe-Pro-D-
Tyr-His-Lys
Glu-Arg-D-Lys-Tyr-D-Val-Phe-D-His-Trp-Arg-D-
Gly-Tyr-Arg-D-Met-NH₂
Arg-D-Leu-D-Tyr-Phe-Lys-Glu-D-Lys-Arg-D-Trp-
Lys-D-Phe-Tyr-D-Arg-Gly
D-Glu-Asp-Lys-D-Arg-D-His-Phe-Phe-D-Val-Tyr-
Arg-Tyr-D-Tyr-Arg-His-Phe-NH₂
Asp-Arg-D-Phe-Cys-Phe-D-Arg-D-Lys-Tyr-Arg-D-
Tyr-Trp-D-His-Tyr-D-Phe-Lys-Phe
His-Tyr-D-Arg-Trp-Lys-Phe-D-Asp-Ala-Arg-Cys-D-
Tyr-His-Phe-D-Lys-Tyr-His-Ser-NH₂
Gly-Ala-Lys-Phe-D-Lys-Glu-Arg-Tyr-His-D-Arg-D-
Arg-Asp-Tyr-Trp-D-His-Trp-His-D-Lys-Asp
Thr-Tyr-Arg-D-Lys-Trp-Tyr-Glu-Asp-D-Lys-D-Arg-
His-Phe-D-Tyr-Gly-Val-Ile-D-His-Arg-Tyr-Lys-NH₂

In one embodiment, the aromatic-cationic peptide has the formula Phe-D-Arg-Phe-Lys-NH₂. In another embodiment, the aromatic-cationic peptide has the formula D-Arg-2′6′-Dmt-Lys-Phe-NH₂.

The peptides mentioned herein and their derivatives can further include functional analogs. A peptide is considered a functional analog if the analog has the same function as the stated peptide. The analog may, for example, be a substitution variant of a peptide, wherein one or more amino acids are substituted by another amino acid. Suitable substitution variants of the peptides include conservative amino acid substitutions. Amino acids may be grouped according to their physicochemical characteristics as follows:

(a) Non-polar amino acids: Ala(A) Ser(S) Thr(T) Pro(P) Gly(G) Cys (C);

(b) Acidic amino acids: Asn(N) Asp(D) Glu(E) Gln(Q);

(c) Basic amino acids: His(H) Arg(R) Lys(K);

(d) Hydrophobic amino acids: Met(M) Leu(L) Ile(I) Val(V); and

(e) Aromatic amino acids: Phe(F) Tyr(Y) Trp(W) His (H).

Substitutions of an amino acid in a peptide by another amino acid in the same group is referred to as a conservative substitution and may preserve the physicochemical characteristics of the original peptide. In contrast, substitutions of an amino acid in a peptide by another amino acid in a different group are generally more likely to alter the characteristics of the original peptide.

Examples of peptides include, but are not limited to, the aromatic-cationic peptides shown in Table 5.

TABLE 5
Peptide Analogs with Mu-Opioid Activity

| Amino Acid | Amino Acid | Amino Acid | Amino Acid | C-Terminal |
|---|---|---|---|---|
| Position 1 | Position 2 | Position 3 | Position 4 | Modification |
| Tyr | D-Arg | Phe | Lys | NH₂ |
| Tyr | D-Arg | Phe | Orn | NH₂ |
| Tyr | D-Arg | Phe | Dab | NH₂ |
| Tyr | D-Arg | Phe | Dap | NH₂ |
| 2′6′Dmt | D-Arg | Phe | Lys | NH₂ |
| 2′6′Dmt | D-Arg | Phe | Lys-NH(CH₂)₂—NH-dns | NH₂ |
| 2′6′Dmt | D-Arg | Phe | Lys-NH(CH₂)₂—NH-atn | NH₂ |
| 2′6′Dmt | D-Arg | Phe | dnsLys | NH₂ |
| 2′6′Dmt | D-Cit | Phe | Lys | NH₂ |
| 2′6′Dmt | D-Cit | Phe | Ahp | NH₂ |
| 2′6′Dmt | D-Arg | Phe | Orn | NH₂ |
| 2′6′Dmt | D-Arg | Phe | Dab | NH₂ |
| 2′6′Dmt | D-Arg | Phe | Dap | NH₂ |
| 2′6′Dmt | D-Arg | Phe | Ahp(2-aminoheptanoic acid) | NH₂ |
| Bio-2′6′Dmt | D-Arg | Phe | Lys | NH₂ |
| 3′5′Dmt | D-Arg | Phe | Lys | NH₂ |
| 3′5′Dmt | D-Arg | Phe | Orn | NH₂ |
| 3′5′Dmt | D-Arg | Phe | Dab | NH₂ |
| 3′5′Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′6′Dmt | D-Arg | Tyr | Lys | NH₂ |
| 2′6′Dmt | D-Arg | Tyr | Orn | NH₂ |
| 2′6′Dmt | D-Arg | Tyr | Dab | NH₂ |
| 2′6′Dmt | D-Arg | Tyr | Dap | NH₂ |
| 2′6′Dmt | D-Arg | 2′6′Dmt | Lys | NH₂ |
| 2′6′Dmt | D-Arg | 2′6′Dmt | Orn | NH₂ |
| 2′6′Dmt | D-Arg | 2′6′Dmt | Dab | NH₂ |
| 2′6′Dmt | D-Arg | 2′6′Dmt | Dap | NH₂ |
| 3′5′Dmt | D-Arg | 3′5′Dmt | Arg | NH₂ |
| 3′5′Dmt | D-Arg | 3′5′Dmt | Lys | NH₂ |
| 3′5′Dmt | D-Arg | 3′5′Dmt | Orn | NH₂ |
| 3′5′Dmt | D-Arg | 3′5′Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′6′Dmt | D-Lys | Phe | Dab | NH₂ |
| 2′6′Dmt | D-Lys | Phe | Dap | NH₂ |
| 2′6′Dmt | D-Lys | Phe | Arg | NH₂ |
| 2′6′Dmt | D-Lys | Phe | Lys | NH₂ |
| 3′5′Dmt | D-Lys | Phe | Orn | NH₂ |
| 3′5′Dmt | D-Lys | Phe | Dab | NH₂ |
| 3′5′Dmt | D-Lys | Phe | Dap | NH₂ |
| 3′5′Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′6′Dmt | D-Lys | Tyr | Lys | NH₂ |
| 2′6′Dmt | D-Lys | Tyr | Orn | NH₂ |
| 2′6′Dmt | D-Lys | Tyr | Dab | NH₂ |
| 2′6′Dmt | D-Lys | Tyr | Dap | NH₂ |
| 2′6′Dmt | D-Lys | 2′6′Dmt | Lys | NH₂ |
| 2′6′Dmt | D-Lys | 2′6′Dmt | Orn | NH₂ |
| 2′6′Dmt | D-Lys | 2′6′Dmt | Dab | NH₂ |
| 2′6′Dmt | D-Lys | 2′6′Dmt | Dap | NH₂ |
| 2′6′Dmt | D-Arg | Phe | dnsDap | NH₂ |
| 2′6′Dmt | D-Arg | Phe | atnDap | NH₂ |
| 3′5′Dmt | D-Lys | 3′5′Dmt | Lys | NH₂ |
| 3′5′Dmt | D-Lys | 3′5′Dmt | Orn | NH₂ |
| 3′5′Dmt | D-Lys | 3′5′Dmt | Dab | NH₂ |
| 3′5′Dmt | D-Lys | 3′5′Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′6′Dmt | D-Arg | Phe | Arg | NH₂ |
| 2′6′Dmt | D-Lys | Phe | Arg | NH₂ |
| 2′6′Dmt | D-Orn | Phe | Arg | NH₂ |
| 2′6′Dmt | D-Dab | Phe | Arg | NH₂ |
| 3′5′Dmt | D-Dap | Phe | Arg | NH₂ |
| 3′5′Dmt | D-Arg | Phe | Arg | NH₂ |
| 3′5′Dmt | D-Lys | Phe | Arg | NH₂ |
| 3′5′Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′6′Dmt | D-Arg | 2′6′Dmt | Arg | NH₂ |
| 2′6′Dmt | D-Lys | 2′6′Dmt | Arg | NH₂ |
| 2′6′Dmt | D-Orn | 2′6′Dmt | Arg | NH₂ |
| 2′6′Dmt | D-Dab | 2′6′Dmt | Arg | NH₂ |
| 3′5′Dmt | D-Dap | 3′5′Dmt | Arg | NH₂ |
| 3′5′Dmt | D-Arg | 3′5′Dmt | Arg | NH₂ |
| 3′5′Dmt | D-Lys | 3′5′Dmt | Arg | NH₂ |
| 3′5′Dmt | D-Orn | 3′5′Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |

Dab = diaminobutyric
Dap = diaminopropionic acid
Dmt = dimethyltyrosine
Mmt = 2′-methyltyrosine
Tmt = N, 2′,6′-trimethyltyrosine
Hmt = 2′-hydroxy,6′-methyltyrosine
dnsDap = β-dansyl-L-α,β-diaminopropionic acid
atnDap = β-anthraniloyl-L-α,β-diaminopropionic acid
Bio = biotin

Examples of peptides also include, but are not limited to, the aromatic-cationic peptides shown in Table 6.

TABLE 6
Peptide Analogs Lacking Mu-Opioid Activity

| Amino | Amino | Amino | Amino | |
|---|---|---|---|---|
| Acid | Acid | Acid | Acid | C-Terminal |
| Position 1 | Position 2 | Position 3 | Position 4 | Modification |
| D-Arg | Dmt | Lys | Phe | NH₂ |
| D-Arg | Dmt | Phe | Lys | NH₂ |
| D-Arg | Phe | Lys | Dmt | NH₂ |
| D-Arg | Phe | Dmt | Lys | NH₂ |
| D-Arg | Lys | Dmt | Phe | NH₂ |
| D-Arg | Lys | Phe | Dmt | NH₂ |
| Phe | Lys | Dmt | D-Arg | NH₂ |
| Phe | Lys | D-Arg | Dmt | NH₂ |
| Phe | D-Arg | Phe | Lys | NH₂ |
| Phe | D-Arg | Dmt | Lys | NH₂ |
| Phe | D-Arg | Lys | Dmt | NH₂ |
| Phe | Dmt | D-Arg | Lys | NH₂ |
| Phe | Dmt | Lys | D-Arg | NH₂ |
| Lys | Phe | D-Arg | Dmt | NH₂ |
| Lys | Phe | Dmt | D-Arg | NH₂ |
| Lys | Dmt | D-Arg | Phe | NH₂ |
| Lys | Dmt | Phe | D-Arg | NH₂ |
| Lys | D-Arg | Phe | Dmt | NH₂ |
| Lys | D-Arg | Dmt | Phe | NH₂ |
| D-Arg | Dmt | D-Arg | Phe | NH₂ |
| D-Arg | Dmt | D-Arg | Dmt | NH₂ |
| D-Arg | Dmt | D-Arg | Tyr | NH₂ |
| D-Arg | Dmt | D-Arg | Trp | NH₂ |
| Trp | D-Arg | Phe | Lys | NH₂ |
| Trp | D-Arg | Tyr | Lys | NH₂ |
| Trp | D-Arg | Trp | Lys | NH₂ |
| Trp | D-Arg | Dmt | Lys | NH₂ |
| D-Arg | Trp | Lys | Phe | NH₂ |
| D-Arg | Trp | Phe | Lys | NH₂ |
| D-Arg | Trp | Lys | Dmt | NH₂ |
| D-Arg | Trp | Dmt | Lys | NH₂ |
| D-Arg | Lys | Trp | Phe | NH₂ |
| D-Arg | Lys | Trp | Dmt | NH₂ |
| Cha | D-Arg | Phe | Lys | NH₂ |
| Ala | D-Arg | Phe | Lys | NH₂ |

Cha = cyclohexyl alanine

The amino acids of the peptides shown in Table 5 and 6 may be in either the L- or the D-configuration.

The peptides may be synthesized by any of the methods well known in the art.

Suitable methods for chemically synthesizing the protein include, for example, those described by Stuart and Young in Solid Phase Peptide Synthesis, Second Edition, Pierce Chemical Company (1984), and in Methods Enzymol., 289, Academic Press, Inc, New York (1997).

### Active Agents

The methods include the use of an aromatic-cationic peptide as described herein together with one or more additional therapeutic agents for the treatment of ischemia-reperfusion injury or AMI. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

In some embodiments, the combination therapy comprises administering to a subject in need thereof an aromatic-cationic peptide composition combined with an active agent selected from the group consisting of an angiotensin converting enzyme (ACE) inhibitor, a beta-blocker, a diuretic, an anti-arrhythmic agent, an anti-anginal agent, a tyrosine kinase receptor agonist, an anticoagulant, and a hypercholesterolemic agent.

In one embodiment, the active agent is an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, lignocaine moricizine, mexiletine, tocainide, procainamide, encainide, flecanide, tocainide, phenytoin, propafenone, quinidine, disopyramide, and flecainide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltaizem, digitalis, adenosine, nickel chloride, and magnesium ions. The effects of an exemplary anti-arrhythmia agent in preventing or treating ischemia-reperfusion injury are described in Mohan et al., Cardioprotection by HO-4038, a novel verapamil derivative, targeted against ischemia and reperfusion-mediated acute myocardial infarction. American Journal of Physiology—Heart & Circulatory Physiology. 296(1): H140-51 (2009).

In one embodiment, the active agent is a vasodilator, for example, bencyclane, cinnarizine, citicoline, cyclandelate, cyclonicate, ebumamonine, hydralazine phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin. The effects of an exemplary vasodilator in preventing or treating ischemia-reperfusion injury are described in Garcia-Gonzalez, et al., New pharmacologic options in the treatment of acute coronary syndromes and myocardial ischemia-reperfusion injury: potential role of levosimendan. Minerva Cardioangiologica. 55(5): 625-35 (2007).

In one embodiment, the active agent is an anti-anginal agent, for example, nitrates, isosorbide nitrate, glyceryl trinitrate and pentaerythritol tetranitrate. The effects of an exemplary anti-anginal agent in preventing or treating ischemia-reperfusion injury are described in Kennedy et al., Effect of perhexiline and oxfenicine on myocardial function and metabolism during low-flow ischemia/reperfusion in the isolated rat heart. Journal of Cardiovascular Pharmacology. 36(6): 794-801 (2000).

In one embodiment, the active agent is a corticosteroid, such as hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. The effects of an exemplary corticosteroid in preventing or treating ischemia-reperfusion injury are described in Varas-Lorenzo et al., Use of oral corticosteroids and the risk of acute myocardial infarction. Atherosclerosis. 192(2): 376-83 (2007).

In one embodiment, the active agent is a cardioglycoside, for example, digoxin and digitoxin.

In one embodiment, the active agent is a diuretic, such as thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, and potassium can renoate), osmotic diuretics (such as isosorbide, D-mannitol, and glycerin), nonthiazide diuretics (such as meticrane, tripamide, chlorthalidone, and mefruside), and acetazolamide. The effects of an exemplary diuretic in preventing or treating ischemia-reperfusion injury are described in Kasama et al., Effects of intravenous atrial natriuretic peptide on cardiac sympathetic nerve activity and left ventricular remodeling in patients with first anterior acute myocardial infarction. Journal of the American College of Cardiology. 49(6):667-74 (2007).

In one embodiment, the active agent is a sedative, for example, nitrazepam, flurazepam and diazepam. The effects of an exemplary sedative in preventing or treating ischemia-reperfusion injury are described in Lucchinetti et al., Sevoflurane inhalation at sedative concentrations provides endothelial protection against ischemia-reperfusion injury in humans. Anesthesiology. 106(2):262-268 (2007).

In one embodiment, the active agent is a cyclooxygenase inhibitor such as aspirin or indomethacin. In one embodiment, the cardiovascular agent is a platelet aggregation inhibitor such as clopidogrel, ticlopidene or aspirin. The effects of an exemplary cyclooxygenase inhibitor in preventing or treating ischemia-reperfusion injury are described in Bassuk et al., Non-selective cyclooxygenase inhibition before periodic acceleration (pGz) cardiopulmonary resuscitation (CPR) in a porcine model of ventricular fibrillation. Resuscitation. 77(2):250-7 (2008).

In one embodiment, the active agent is a angiotensin converting enzyme (ACE) inhibitor such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril, and salts of such compounds. The effects of an exemplary ACE inhibitor in preventing or treating ischemia-reperfusion injury are described in Kingma, J. H. and van Gilst, W. H., Angiotensin-converting enzyme inhibition during thrombolytic therapy in acute myocardial infarction: the Captopril and Thrombolysis Study (CATS). Herz. 18 Suppl 1:416-23 (1993).

In one embodiment, the active agent is an angiotensin II antagonist such as losartan, candesartan, valsartan, eprosartan, and irbesartan. The effects of an exemplary angiotensin II antagonist in preventing or treating ischemia-reperfusion injury are described in Moller et al., Effects of losartan and captopril on left ventricular systolic and diastolic function after acute myocardial infarction: results of the Optimal Trial in Myocardial Infarction with Angiotensin II Antagonist Losartan (OPTIMAAL) echocardiographic substudy. American Heart Journal. 147(3):494-501 (2004).

In one embodiment, the active agent is a thrombolytic agent such as tissue-type plasminogen activators (such as alteplase, tisokinase, nateplase, pamiteplase, monteplase, and rateplase), nasaruplase, streptokinase, urokinase, prourokinase, and anisoylated plasminogen streptokinase activator complex (APSAC, Eminase, Beecham Laboratories), aspirin, heparin, and Warfarin that inhibits Vit K-dependent factors, low molecular weight heparins that inhibit factors X and II, thrombin inhibitors, inhibitors of platelet GP IIbIIIa receptors, inhibitors of tissue factor (TF), inhibitors of human von Willebrand factor, reptilase, TNK-t-PA, staphylokinase, or animal salivary gland plasminogen activators. The effects of an exemplary thrombolytic agent in preventing or treating ischemia-reperfusion injury are described in Sikri, N. and Bardia, A., A history of streptokinase use in acute myocardial infarction. Texas Heart Institute Journal. 34(3):318-27 (2007).

In one embodiment, the active agent is a calcium channel blocking agent such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline. The effects of an exemplary calcium channel blocking agent dilitazem in preventing or treating ischemia-reperfusion injury are described in Fansa et al., Does diltiazem inhibit the inflammatory response in cardiopulmonary bypass? Medical Science Monitor. 9(4):PI30-6 (2003).

In one embodiment, the active agent is a thromboxane receptor antagonist such as ifetroban, prostacyclin mimetics, or phosphodiesterase inhibitors. The effects of an exemplary thromboxane receptor antagonist in preventing or treating ischemia-reperfusion injury are described in Viehman et al., Daltroban, a thromboxane receptor antagonist, protects the myocardium against reperfusion injury following myocardial ischemia without protecting the coronary endothelium. Methods & Findings in Experimental & Clinical Pharmacology. 12(10):651-6 (1990).

In one embodiment, the active agent is a radical scavenger, such as edaravone, vitamin E, and vitamin C. The effects of an exemplary radical scavenger in preventing or treating ischemia-reperfusion injury are described in Higashi et al., Edaravone (3-methyl-1-phenyl-2-pyrazolin-5-one), a novel free radical scavenger, for treatment of cardiovascular diseases. Recent Patents on Cardiovascular Drug Discovery. 1(1):85-93 (2006).

In one embodiment, the active agent is a antiplatelet drug, such as ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene. The effects of an exemplary antiplatelet drug in preventing or treating ischemia-reperfusion injury are described in Ochiai et al., Impact of cilostazol on clinical and angiographic outcome after primary stenting for acute myocardial infarction. American Journal of Cardiology. 84(9):1074-6, A6, A9, (1999).

In one embodiment, the active agent is a β-adrenaline receptor blocking drug, such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol. The effects of an exemplary β-adrenaline receptor blocking drug in preventing or treating ischemia-reperfusion injury are described in Kovacs et al., Prevalent role of Akt and ERK activation in cardioprotective effect of Ca(2+) channel- and beta-adrenergic receptor blockers. Molecular & Cellular Biochemistry. 321(1-2):155-164 (2009).

In one embodiment, the active agent is a α-receptor blocking drug, such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine. The effects of an exemplary α-receptor blocking drug in preventing or treating ischemia-reperfusion injury are described in Kim et al., Involvement of adrenergic pathways in activation of catalase by myocardial ischemia-reperfusion. American Journal of Physiology—Regulatory Integrative & Comparative Physiology. 282(5):R1450-1458, (2002).

In one embodiment, the active agent is an inotrope. Positive inotropic agents increase myocardial contractility, and are used to support cardiac function in conditions such as decompensated congestive heart failure, cardiogenic shock, septic shock, myocardial infarction, cardiomyopathy, etc. Examples of positive inotropic agents include, but are not limited to, Berberine, Bipyridine derivatives, Inamrinone, Milrinone, Calcium, Calcium sensitizers, Levosimendan, Cardiac glycosides, Digoxin, Catecholamines, Dopamine, Dobutamine, Dopexamine, Epinephrine (adrenaline), Isoprenaline (isoproterenol), Norepinephrine (noradrenaline), Eicosanoids, Prostaglandins, Phosphodiesterase inhibitors, Enoximone, Milrinone, Theophylline, and Glucagon. Negative inotropic agents decrease myocardial contractility, and are used to decrease cardiac workload in conditions such as angina. While negative inotropism may precipitate or exacerbate heart failure, certain beta blockers (e.g. carvedilol, bisoprolol and metoprolol) have been shown to reduce morbidity and mortality in congestive heart failure. Examples of negative inotropic agents include, but are not limited to, Beta blockers, Calcium channel blockers, Diltiazem, Verapamil, Clevidipine, Quinidine, Procainamide, disopyramide, and Flecainide.

In one embodiment, the active agent is a sympathetic nerve inhibitor, such as clonidine, guanfacine, guanabenz, methyldopa, and reserpine, hydralazine, todralazine, budralazine, and cadralazine. The effects of an exemplary sympathetic nerve inhibitor in preventing or treating ischemia-reperfusion injury are described in Chamberlain, D. A. and Vincent, R., Combined receptor intervention and myocardial infarction. Drugs. 28 Suppl 2:88-108, (1984).

In one embodiment, the active agent is a digitalis formulation such as digitoxin, digoxin, methyldigoxin, deslanoside, vesnarinone, lanatoside C, and proscillaridin. The effects of an exemplary digitalis formulation in preventing or treating ischemia-reperfusion injury are described in Sanazaro, P. J., Use of deslanoside in acute myocardial infarction and cardiac emergencies: a probative agent for assessing digitalis saturation and for intramuscular digitalization. American Practitioner & Digest of Treatment. 8(12):1933-41, (1957).

In one embodiment, the active agent is an antihyperlipidemic drug, such as atorvastatin, simvastatin, pravastatin sodium, fluvastatin sodium, clinofibrate, clofibrate, simfibrate, fenofibrate, bezafibrate, colestimide, and colestyramine. The effects of an exemplary antihyperlipidemic drug in preventing or treating ischemia-reperfusion injury are described in Ye et al., Enhanced cardioprotection against ischemia-reperfusion injury with a dipyridamole and low-dose atorvastatin combination. American Journal of Physiology—Heart & Circulatory Physiology. 293(1):H813-8 (2007).

### Therapeutic Uses of Aromatic-Cationic Peptides and Cardiovascular Agents

General.

The aromatic-cationic peptides described herein are useful to prevent or treat disease. The combination of peptides and active agents described above are useful in treating any ischemia and/or reperfusion of a tissue or organ. Ischemia in a tissue or organ of a mammal is a multifaceted pathological condition which is caused by oxygen deprivation (hypoxia) and/or glucose (e.g., substrate) deprivation. Oxygen and/or glucose deprivation in cells of a tissue or organ leads to a reduction or total loss of energy generating capacity and consequent loss of function of active ion transport across the cell membranes. Oxygen and/or glucose deprivation also leads to pathological changes in other cell membranes, including permeability transition in the mitochondrial membranes. In addition other molecules, such as apoptotic proteins normally compartmentalized within the mitochondria, may leak out into the cytoplasm and cause apoptotic cell death. Profound ischemia can lead to necrotic cell death.

Ischemia or hypoxia in a particular tissue or organ may be caused by a loss or severe reduction in blood supply to the tissue or organ. The loss or severe reduction in blood supply may, for example, be due to thromboembolic stroke, coronary atherosclerosis, or peripheral vascular disease. The tissue affected by ischemia or hypoxia is typically muscle, such as cardiac, skeletal, or smooth muscle. The organ affected by ischemia or hypoxia may be any organ that is subject to ischemia or hypoxia. Examples of organs affected by ischemia or hypoxia include brain, heart, kidney, and prostate. For instance, cardiac muscle ischemia or hypoxia is commonly caused by atherosclerotic or thrombotic blockages which lead to the reduction or loss of oxygen delivery to the cardiac tissues by the cardiac arterial and capillary blood supply. Such cardiac ischemia or hypoxia may cause pain and necrosis of the affected cardiac muscle, and ultimately may lead to cardiac failure. Ischemia or hypoxia in skeletal muscle or smooth muscle may arise from similar causes. For example, ischemia or hypoxia in intestinal smooth muscle or skeletal muscle of the limbs may also be caused by atherosclerotic or thrombotic blockages.

Reperfusion is the restoration of blood flow to any organ or tissue in which the flow of blood is decreased or blocked. For example, blood flow can be restored to any organ or tissue affected by ischemia or hypoxia. The restoration of blood flow (reperfusion) can occur by any method known to those in the art. For instance, reperfusion of ischemic cardiac tissues may arise from angioplasty, coronary artery bypass graft, or the use of thrombolytic drugs.

In some embodiments, a pharmaceutical composition comprising an aromatic-cationic peptide and a second active agent are administered to a subject suffering from ischemia and/or reperfusion injury of the brain, heart, kidney, prostate, or other organ/tissue susceptible to ischemia and/or reperfusion injury. The aromatic-cationic peptide and a second active agent may be administered separately, sequentially, or simultaneously in effective amounts to reduce or ameliorate the effects of the ischemia and/or reperfusion injury of the brain, heart, kidney, prostate, or other organ/tissue.

The disclosure also provides for both prophylactic and therapeutic methods of treating a subject at risk of (or susceptible to) vessel occlusion injury or cardiac ischemia-reperfusion injury. Accordingly, the present methods provide for the prevention and/or treatment of vessel occlusion injury or cardiac ischemia-reperfusion injury in a subject by administering an effective amount of an aromatic-cationic peptide and one or more cardiovascular agents to a subject in need thereof

In various embodiments, suitable in vitro or in vivo assays are performed to determine the effect of a specific combination of aromatic-cationic peptides and one or more active agents and whether its administration is indicated for treatment. In various embodiments, assays can be performed with representative animal models to determine if a given aromatic-cationic peptide and cardiovascular agent treatment regime exerts the desired effect in preventing or treating ischemia-reperfusion injury. Compounds for use in therapy can be tested in suitable animal model systems including, but not limited to rats, mice, chicken, pigs, cows, monkeys, rabbits, and the like, prior to testing in human subjects. Any of the animal model systems known in the art can be used prior to administration to human subjects.

In therapeutic applications, compositions or medicaments are administered to a subject suspected of, or already suffering from such a disease in an amount sufficient to cure, or at least partially arrest, the symptoms of the disease, including its complications and intermediate pathological phenotypes in development of the disease. As such, the present technology provides methods of treating an individual afflicted with cardiac ischemia-reperfusion injury.

### Modes of Administration and Effective Dosages

Any method known to those in the art for contacting a cell, organ or tissue with a peptide and active agent may be employed. Suitable methods include in vitro, ex vivo, or in vivo methods. In vivo methods typically include the administration of an aromatic-cationic peptide and active agent, such as those described above, to a mammal, suitably a human. When used in vivo for therapy, the aromatic-cationic peptides and active agents are administered to the subject in effective amounts (i.e., amounts that have desired therapeutic effect). The dose and dosage regimen will depend upon the degree of the injury in the subject, the characteristics of the particular aromatic-cationic peptide used, e.g., its therapeutic index, the subject, and the subject's history.

The effective amount may be determined during pre-clinical trials and clinical trials by methods familiar to physicians and clinicians. An effective amount of a peptide and active agent useful in the methods may be administered to a mammal in need thereof by any of a number of well-known methods for administering pharmaceutical compounds. The peptide may be administered systemically or locally.

The compound may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the pharmaceutically acceptable salt is acetate or trifluoroacetate salt.

The compounds described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof

Oral compositions generally include an inert diluent or an edible carrier. For the purpose of oral therapeutic administration, the active compounds can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the aromatic-cationic peptides and/or cardiovascular agents, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.1-10,000 micrograms per kg body weight. In one embodiment, aromatic-cationic peptide concentrations in a carrier range from 0.2 to 2000 micrograms per delivered milliliter.

In some embodiments, a therapeutically effective amount of an aromatic-cationic peptide may be defined as a concentration of peptide at the target tissue of 10⁻¹¹ to 10⁻⁶ molar, e.g., approximately 10⁻⁷ molar. This concentration may be delivered by systemic doses of 0.01 to 100 mg/kg or equivalent dose by body surface area. The schedule of doses would be optimized to maintain the therapeutic concentration at the target tissue, most preferably by single daily or weekly administration, but also including continuous administration (e.g., parenteral infusion or transdermal application).

In some embodiments, the dosage of the aromatic-cationic peptide is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h. In an illustrative embodiment, the dose of cardiovascular agent is from about 1 to 100 mg/kg, suitably about 25 mg/kg.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In a preferred embodiment, the mammal is a human.

### EXAMPLES

The present technology is further illustrated by the following example, which should not be construed as limiting in any way.

### Example 1

### Effects of an Aromatic-Cationic Peptide in Protecting Against Acute Myocardial Infarction Injury in a Rabbit Model

The effects of a combination of aromatic-cationic peptides and a cardiovascular agent in protecting against an acute myocardial infarction injury in a rabbit model are investigated.

New Zealand white rabbits are used in this study. The rabbits are males and >10 weeks in age. Environmental controls in the animal rooms are set to maintain temperatures of 61° to 72° F. and relative humidity between 30% and 70%. Room temperature and humidity are recorded hourly, and monitored daily. There are approximately 10-15 air exchanges per hour in the animal rooms. Photoperiod is 12-hr light/12-hr dark (via fluorescent lighting) with exceptions as necessary to accommodate dosing and data collection. Routine daily observations are performed. Harlan Teklad, Certified Diet (2030C), rabbit diet is provided approximately 180 grams per day from arrival to the facility. In addition, fresh fruits and vegetables are given to the rabbit 3 times a week.

The peptide D-Arg-2′6′-Dmt-Lys-Phe-NH₂ (sterile lyophilized powder) is used as the peptide test article. Dosing solutions for the peptide are formulated at no more than 1 mg/ml, and are delivered via continuous infusion (IV) at a constant rate (e.g., 50 μL/kg/min). The cardiovascular agent is selected from the group consisting of: an anti-arrhythmia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a thromboxane receptor antagonist, a radical scavenger, an anti-platelet drug, a β-adrenaline receptor blocking drug, α-receptor blocking drug, a sympathetic nerve inhibitor, a digitalis formulation, and an antihyperlipidemic drug. The dose is selected based on known effective dosages for the compounds of interest. Normal saline (0.9% NaCl) is used as a control.

The test/vehicle articles are given intravenously, under general anesthesia, in order to mimic the expected route of administration in the clinical setting of AMI and PTCA. Intravenous infusion is administered via a peripheral vein using a Kd Scientific infusion pump (Holliston, Mass. 01746) at a constant volume (e.g., 50 μL/kg/min).

The study follows a predetermined placebo and sham controlled design. In short, 10-20 healthy, acclimatized, male rabbits are assigned to one of three study arms (approximately 2-10 animals per group in each arm). Arm A (CTRL/PLAC) includes animals treated with vehicle (vehicle; VEH, IV); Arm B (treated) includes animals treated with peptide, peptide+cardiovascular agent, or cardiovascular agent; Arm C (SHAM) includes sham-operated (surgery) time-controls treated with vehicle (vehicle; VEH, IV) or peptide.

TABLE 7
Study Design.

| | | Ischemia Time | Reperfusion Time |
|---|---|---|---|
| Group | Study Group | (administration protocol) | evaluated |
| A | CONTROL/PLACEBO | 30 minute total ischemia | 180 minutes |
| | BOLUS | (Start placebo after 10 minutes of | |
| | | ischemia such that the last 20 | |
| | | minutes of ischemia include | |
| | | placebo administration. | |
| | | Administer a bolus injection of | |
| | | placebo immediately prior to | |
| | | release of coronary snare. | |
| | | Continue administering placebo | |
| | | through reperfusion) | |
| B1 | PEPTIDE + | 30 minute total | 180 minutes |
| | Cardiovascular Agent | (Start peptide, cardiovascular | |
| | BOLUS | agent (CVA) or peptide + CVA | |
| | | after 10 minutes of ischemia such | |
| | | that the last 20 minutes of | |
| | | ischemia include peptide, CVA or | |
| | | peptide + CVA administration. | |
| | | Administer a bolus of peptide + | |
| | | CVA or a bolus of peptide, or a | |
| | | bolus of CVA immediately prior | |
| | | to release of coronary snare. | |
| | | Continue administering peptide + | |
| | | CVA, peptide or CVA through | |
| | | reperfusion) | |
| B2 | PEPTIDE + | 30 minute total | 180 minutes |
| | Cardiovascular Agent | (Start peptide, CVA or peptide + | |
| | | CVA after 10 minutes of ischemia | |
| | | such that the last 20 minutes of | |
| | | ischemia include peptide, CVA or | |
| | | peptide + CVA administration. | |
| | | Release coronary snare— | |
| | | no bolus administration prior to | |
| | | reperfusion. Continue | |
| | | administering peptide + CVA, | |
| | | peptide or CVA through | |
| | | reperfusion) | |
| C | SHAM | 0 Min | 180 minutes |
| | (FOR SURGERY | (Start peptide + cardiovascular | |
| | WITHOUT ISCHEMIA) | agent (CVA) after 10 minutes of | |
| | | sham surgery such that the last 20 | |
| | | minutes of sham surgery include | |
| | | placebo or peptide administration. | |
| | | Administer a bolus of placebo or | |
| | | peptide after 30 minutes of sham | |
| | | surgery. Continue administering | |
| | | placebo or peptide through | |
| | | reperfusion) | |

In all cases, treatments are started approximately 10 min after the onset of a 30 min ischemic insult (coronary occlusion) and continued for up to 3 h following reperfusion. In all cases, cardiovascular function is monitored both prior to and during ischemia, as well as for up to 180 min (3 h) post-reperfusion. The experiments are terminated 3 h post-reperfusion (end of study); irreversible myocardial injury (infarct size by histomorphometery) at this time-point is evaluated, and is the primary-end-point of the study.

Anesthesia/Surgical Preparation.

General anesthesia is induced intramuscularly (IM) with a ketamine (˜35-50 mg/kg)/xylazine (˜5-10 mg/kg) mixture. A venous catheter is placed in a peripheral vein (e.g., ear) for the administration of anesthetics. In order to preserve autonomic function, anesthesia is maintained with continuous infusions of propofol (˜8-30 mg/kg/hour) and ketamine (˜1.2-2.4 mg/kg/hr). A cuffed tracheal tube is placed via a tracheotomy (ventral midline incision) and used to mechanically ventilate the lungs with a 95% O₂/5% CO₂ mixture via a volume-cycled animal ventilator (˜40 breaths/minute with a tidal volume of ˜12.5 ml/kg) in order to sustain PaCO₂ values broadly within the physiological range.

Once a surgical plane of anesthesia is reached, either transthoracic or needle electrodes forming two standard ECG leads (e.g., lead II, aVF, V2) are placed. A cervical cut-down exposes a carotid artery, which is isolated, dissected free from the surrounding tissue and cannulated with a dual-sensor high-fidelity micromanometer catheter (Millar Instruments); the tip of this catheter is advanced into the left-ventricle (LV) retrogradely across the aortic valve, in order to simultaneously determine aortic (root, proximal transducer) and left-ventricular (distal transducer) pressures. The carotid cut-down also exposes the jugular vein, which is cannulated with a hollow injection catheter (for blood sampling). Finally, an additional venous catheter is placed in a peripheral vein (e.g., ear) for the administration of vehicle/test articles.

Subsequently, the animals are placed in right-lateral recumbence and the heart is exposed via a midline thoracotomy and a pericardiotomy. The heart is suspended on a pericardial cradle in order to expose the left circumflex (LCX) and the left-anterior descending (LAD) coronary arteries. Silk ligatures are loosely placed (using a taper-point needle) around the proximal LAD and if necessary, depending on each animal's coronary anatomy, around one or more branches of the LCX marginal coronary arteries. Tightening of these snares (via small pieces of polyethylene tubing) allows rendering a portion of the left ventricular myocardium temporarily ischemic.

Once instrumentation is completed, hemodynamic stability and proper anesthesia depth are verified/ensured for at least 30 min. Subsequently, the animals are paralyzed with atracurium (˜0.1 to 0.2 mg/kg/hr IV) in order to facilitate hemodynamic/respiratory stability. Following atracurium administration, signs of autonomic hyperactivity and/or changes in BIS values are used to evaluate anesthesia depth and/or to up-titrate the intravenous anesthetics.

Experimental Protocol/Cardiovascular Data Collection.

Immediately following surgical preparation, the animals are heparinized (100 units heparin/kg/h, IV bolus), and after hemodynamic stabilization (for approximately 30 min), baseline data are collected including venous blood for the evaluation of cardiac enzymes/biomarkers as well as of test-article concentrations.

Following hemodynamic stabilization and baseline measurements, the animals are subjected to an acute 60 min ischemic insult by tightening of the LAD/LCX coronary artery snares. Myocardial ischemia is visually confirmed by color (i.e., cyanotic) changes in distal distributions of the LAD/LCX and by the onset of electrocardiographic changes. Approximately after 10 min of ischemia, the animals receive a continuous infusion of either vehicle (saline), peptide or peptide+cardiovascular agent; ischemia is continued for an additional 20 min (i.e., 30 min total) after the start of treatment. Subsequently (i.e., after 30 min of ischemia of which the last 20 min overlap with the treatment), the animals receive a bolus dose of cardiovascular agent, peptide, cardiovascular agent plus peptide or vehicle, and the coronary snares are released. The previously ischemic myocardium is reperfused for up to 3 h. Treatment with either vehicle, peptide, cardiovascular agent or cardiovascular agent plus peptide is continued throughout the reperfusion period. It should be noted that in sham-operated animals the vessel snares are manipulated at the time of ischemia/reperfusion onset, but are not either tightened or loosened.

Cardiovascular data collection occurs at 11 pre-determined time-points: post-instrumentation/stabilization (i.e., baseline), after 10 and 30 min of ischemia, as well as at 5, 15, 30, 60, 120, and 180 min post-reperfusion. Throughout the experiments, analog signals are digitally sampled (1000 Hz) and recorded continuously with a data acquisition system (IOX; EMKA Technologies), and the following parameters are determined at the above-mentioned time-points: (1) from bipolar transthoracic ECG (e.g., Lead II, aVF): rhythm (arrhythmia quantification/classification), RR, PQ, QRS, QT, QTc, short-term QT instability, and QT:TQ (restitution); (2) from solid-state manometer in aorta (Millar): arterial/aortic pressure (AoP); and (3) from solid-state manometer in the LV (Millar): left-ventricular pressures (ESP, EDP) and derived indices (dP/dtmax, dP/dtmin, Vmax, and tau). In addition, in order to determine/quantify the degree of irreversible myocardial injury (i.e., infarction) resulting from the I/R insult with and without peptide treatment, cardiac biomarkers as well as infarct area are evaluated.

Blood Samples.

Venous (<3 mL) whole blood samples are collected for both pharmaco-kinetic (PK) analysis as well as for the evaluation of myocardial injury via cardiac biomarker analyses at six data-collection time-points: baseline, 30 min of ischemia, as well as 30, 60, 120 and 180 min post-reperfusion. Two clinically used biomarkers are measured: cardiac Troponin-I (cTnI) and creatine-kinase (CK-MB). In addition, three arterial (˜0.5 mL) whole blood samples are collected at baseline, 30 min of ischemia, as well as the 60 and 180 min post-reperfusion for the determination of blood-gases; the arterial samples are collected into blood gas syringes and used for the measurement of blood-gases via an I-Stat analyzer/cartridges (CG4+).

Histopathology/Histomorphometery.

At the completion of the protocol, irreversible myocardial injury (i.e., infarction) resulting from the I/R insult is evaluated. In short, the coronary snares are retightened and Evan's blue dye (1 mL/kg; Sigma, St. Louis, Mo.) is injected intravenously to delineate the myocardial area-at-risk (AR) during ischemia. Approximately 5 min later, the heart is arrested (by an injection of potassium chloride into the left atrium), and freshly excised. The LV is sectioned perpendicular to its long axis (from apex to base) into 3 mm thick slices. Subsequently, the slices are incubated for 20 min in 2% triphenyl-tetrazolium-chloride (TTC) at 37° C. and fixed in a 10% non-buffered formalin solution (NBF).

Following fixation, the infarct and at-risks areas are delineated/measured digitally. For such purpose, the thickness of each slice is measured with a digital micrometer and later photographed/scanned. All photographs are imported into an image analysis program (Image J; National Institutes of Health), and computer-assisted planometry is performed to determine the overall size of the infarct (I) and at-risk (AR) areas. For each slide, the AR (i.e., not stained blue) is expressed as a percentage of the LV area, and the infarct size (I, not stained tissue) is expressed as a percentage of the AR (I/AR). In all cases, quantitative histomorphometery is performed by personnel blinded to the treatment assignment/study-design.

Animal Observations.

Data are acquired on the EMKA's IOX system using ECG Auto software for analysis (EMKA Technologies). Measurements for all physiological parameters are made manually or automatically from (digital) oscillograph tracings. The mean value from 60 s of data from each targeted time point is used (if possible); however, as mentioned above, signals/tracing are recorded continuously throughout the experiments, in order to allow (if needed) more fine/detailed temporal data analysis (via amendments). Additional calculations are performed using Microsoft Excel. Data is presented as means with standard errors.

It is predicted that infarct size and apoptotic cell death in the peptide or peptide+agent-treated groups will be significantly reduced compared to the control (vehicle alone) group, and that the combination therapy (peptide plus cardiovascular agent) will show improved results as compared to peptide treatment alone or cardiovascular agent alone. These results will indicate that either peptide administration, or a combination of peptide and cardiovascular agent administration prevents the occurrence of acute cardiac ischemia-reperfusion injury. As such, aromatic-cationic peptides, and combination therapy including aromatic-cationic peptide and cardiovascular agents are useful in methods treating ischemia-reperfusion injury in mammalian subjects.

While the present example describes the use of the peptide D-Arg-2′6′-Dmt-Lys-Phe-NH₂ it is understood that other aromatic-cationic peptides disclosed herein could be tested and used with similar results.

### Example 2

### Effects of Combined Peptide and Cardiovascular Agent in a Large Animal Model of Acute Myocardial Infarction Injury

The effects of aromatic-cationic peptides and cardiovascular agent in protecting against cardiac ischemia-reperfusion injury in a large animal model (e.g., a porcine or ovine model) are investigated. The myocardial protective effect of the D-Arg-2′6′-Dmt-Lys-Phe-NH₂ peptide and a cardiovascular agent will be demonstrated by this Example.

General Surgical Protocol for Large Animal Models.

The animals are sedated with intramuscular ketamine (50 mg/kg), glycopyrrolate (0.2 mg/kg), and buprenorphine (0.05 mg/kg). After intubation, animals are ventilated with a mechanical respirator (Hallowell EMC Model AWS; Hallowell, Pittsfield, Mass.) using room air enriched with 0.6 L/min oxygen. Catheters are introduced into a small auricular artery and vein, and into the right jugular vein for the continuous measurement of blood pressure and the administration of intravenous medications. Anesthesia is maintained with an intravenous infusion of ketamine (0.02 to 0.04 mg/kg/min) and supplemental pentothal (2.5 to 5 mg/kg) as needed. Additionally, a pressure transducer (SPR-524; Millar Instruments, Houston, Tex.) is introduced through the right carotid artery into the left ventricle. Heart rate, blood pressure, surface electrocardiogram, and rectal temperature are continuously monitored (Hewlett Packard 78534C; Palo Alto, Calif.).

A left thoracotomy is performed, and a coronary snare is constructed by passing a suture around a large branch of the circumflex coronary artery at approximately 50% of the distance from base to apex of the heart, and threaded through a small piece of polyethylene tubing.

Alternate Surgical Protocol Using an Ovine Model.

Dorset male hybrid sheep weighing 35-40 kg are used in this study. Anesthesia is induced with thiopental sodium (10-15 mg/kg iv), and sheep are intubated, anesthetized with isoflurane (1.5-2%), and ventilated with oxygen (Drager anesthesia monitor, North American Drager, Telford, Pa.). Fluid-filled catheters are placed in a femoral artery and internal jugular vein for the continuous measurement of blood pressure and the administration of intravenous medications. A Swan-Ganz catheter (131h-7F, Baxter Healthcare, Irvine, Calif.) is introduced into the pulmonary artery through the internal jugular vein.

Animals undergo a left thoracotomy, and silicone vascular loops (Quest Medical, Allen, Tex.) are placed around the left anterior descending artery and its second diagonal branch, which is 40% of the distance from the apex to the base of the heart. Occlusion of these arteries at these locations produces a well-characterized model of anteroapical myocardial infarction. Arterial blood pressure, heart rate, surface electrocardiograms (ECG), and rectal temperature are continuously monitored (Hewlett Packard 78534C; Palo Alto, Calif.) throughout the protocol in all animals. A hyper/hypothermia unit (Medi-Therm III, Gaymar Industries, Orchard Park, N.Y.) is used to maintain core temperature of 39-40° C. in sheep. Arterial blood gases are measured in all animals, and the mean pH is maintained at 7.40±0.04 throughout the protocol.

Alternate Surgical Protocol Using a Porcine Model.

Anesthesia is induced in domestic pigs with thiopental sodium (10-15 mg/kg iv), and pigs are intubated, anesthetized with isoflurane (1.5-2%), and ventilated with oxygen (Drager anesthesia monitor, North American Drager, Telford, Pa.). Fluid-filled catheters are placed in a femoral artery and internal jugular vein for the continuous measurement of blood pressure and the administration of intravenous medications. A Swan-Ganz catheter (131h-7F, Baxter Healthcare, Irvine, Calif.) is introduced into the pulmonary artery through the internal jugular vein.

Animals undergo a left thoracotomy, and silicone vascular loops (Quest Medical, Allen, Tex.) are placed around the left anterior descending artery and its second diagonal branch, which is 40% of the distance from the apex to the base of the heart. Occlusion of these arteries at these locations produces a well-characterized model of anteroapical myocardial infarction. Arterial blood pressure, heart rate, surface electrocardiograms (ECG), and rectal temperature are continuously monitored (Hewlett Packard 78534C; Palo Alto, Calif.) throughout the protocol in all animals. A hyper/hypothermia unit (Medi-Therm III, Gaymar Industries, Orchard Park, N.Y.) is used to maintain core temperature of 39-40° C. in the pigs. Arterial blood gases are measured in all animals, and the mean pH is maintained at 7.40±0.04 throughout the protocol.

Treatment Groups.

In the case of the sheep or pig model, animals are divided into six groups, as shown in Table 8 below. The number of animals in each group may be from about 2 to about 15, suitably from about 4 to about 8 animals. After instrumentation, baseline hemodynamic data are recorded. Next, animals receive a 1-hour, continuous 20-mL infusion of either a phosphate buffered saline (PBS) vehicle (control) or peptide (low, mid, or high dose, and cardiovascular agent). The peptide and cardiovascular agent are dissolved in a vehicle. The cardiovascular agent is selected from the group consisting of: an anti-arrhythmia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a thromboxane receptor antagonist, a radical scavenger, an anti-platelet drug, a β-adrenaline receptor blocking drug, α-receptor blocking drug, a sympathetic nerve inhibitor, a digitalis formulation, and an antihyperlipidemic drug. The dose is selected based on known effective dosages for the compounds of interest.

Coronary snares are tightened to produce an ischemic region of the left ventricle. Ischemia is confirmed by a visible color change in the ischemic myocardial region, ST elevations on the electrocardiogram, and regional wall motion abnormalities on echocardiogram. At the end of the 20-120 min ischemic period (preferably 30-60 min), coronary snares are loosened and the previously ischemic myocardium is reperfused for 3 hours. Hemodynamic measurements are recorded throughout the reperfusion period. Each group receives continuous infusion of either a saline vehicle or peptide, as in the exemplary treatment groups shown in Table 8. Variations in the protocol design are contemplated by the present disclosure.

TABLE 8
Treatment Groups

| TREATMENT | # OF | ISCHEMIA PERIOD | | REPERFUSION PERIOD | |
|---|---|---|---|---|---|
| ARM | ANIMALS | DURATION | INTERVENTION | DURATION | INTERVENTION |
| Placebo for | N = 2 | 0 | SHAM for surgery and | 0 | SHAM with placebo for |
| Peptide/Placebo | | | ischemia. | | peptide cont. infusion |
| for Agent | | | Placebo for peptide | | for 180 min |
| | | | administered as | | |
| | | | continuous infusion | | |
| | | | beginning at T + 40 min. | | |
| | | | and ongoing for 20 min. | | |
| | | | Placebo for agent | | |
| | | | administered as bolus | | |
| | | | dose at T + 60 min. | | |
| Peptide/Agent | N = 2 | 0 | SHAM for surgery and | 0 | SHAM with peptide |
| (mid dose) | | | ischemia | | cont. infusion for 180 min |
| | | | Peptide administered as | | |
| | | | continuous infusion | | |
| | | | beginning at T + 40 min. | | |
| | | | and ongoing for 20 min. | | |
| | | | Agent administered as | | |
| | | | bolus dose at T + 60 min. | | |
| Placebo for | N = 8 | 60 min | Placebo for peptide | 180 min | Placebo for peptide |
| Peptide/Placebo | | | administered as | | cont. infusion for 180 min |
| for Agent | | | continuous infusion | | |
| | | | beginning at T + 40 min. | | |
| | | | and ongoing for 20 min. | | |
| | | | Placebo for agent | | |
| | | | administered as bolus | | |
| | | | dose at T + 60 min. | | |
| Placebo for | N = 8 | 60 min | Placebo for peptide | 180 min | Placebo for peptide |
| Peptide/Agent | | | administered as | | cont. infusion for 180 min |
| | | | continuous infusion | | |
| | | | beginning at T + 40 min. | | |
| | | | and ongoing for 20 min. | | |
| | | | Agent administered as | | |
| | | | bolus dose at T + 60 min. | | |
| Peptide/Placebo | N = 8 | 60 min | Peptide administered as | 180 min | Placebo for peptide |
| for Agent | | | continuous infusion | | cont. infusion for 180 min |
| | | | beginning at T + 40 min. | | |
| | | | and ongoing for 20 min. | | |
| | | | Placebo for agent | | |
| | | | administered as bolus | | |
| | | | dose at T + 60 min. | | |
| Peptide (low | N = 8 | 60 min | Peptide administered as | 180 min | Peptide cont. infusion |
| dose)/Agent | | | continuous infusion | | for 180 min |
| | | | beginning at T + 40 min. | | |
| | | | and ongoing for 20 min. | | |
| | | | Agent administered as | | |
| | | | bolus dose at T + 60 min. | | |
| Peptide (mid | N = 8 | 60 min | Peptide administered as | 180 min | Peptide cont. infusion |
| dose)/Agent | | | continuous infusion | | for 180 min |
| | | | beginning at T + 40 min. | | |
| | | | and ongoing for 20 min. | | |
| | | | Agent administered as | | |
| | | | bolus dose at T + 60 min. | | |
| Peptide (high | N = 8 | 60 min | Peptide administered as | 180 min | Peptide cont. infusion |
| dose)/Agent | | | continuous infusion | | for 180 min |
| | | | beginning at T + 40 min. | | |
| | | | and ongoing for 20 min. | | |
| | | | Agent administered as | | |
| | | | bolus dose at T + 60 min. | | |
| Peptide (low | N = 8 | 60 min | Peptide administered | 180 min | Peptide cont. infusion |
| dose)/Agent | | | prior to ischemia, and | | for 180 min |
| | | | as continuous infusion | | |
| | | | beginning at T + 40 min. | | |
| | | | and ongoing for 20 min. | | |
| | | | Agent administered as | | |
| | | | bolus dose at T + 60 min. | | |
| Peptide (mid | N = 8 | 60 min | Peptide administered | 180 min | Peptide cont. infusion |
| dose)/Agent | | | prior to ischemia and as | | for 180 min |
| | | | continuous infusion | | |
| | | | beginning at T + 40 min. | | |
| | | | and ongoing for 20 min. | | |
| | | | Agent administered as | | |
| | | | bolus dose at T + 60 min. | | |
| Peptide (high | N = 8 | 60 min | Peptide administered | 180 min | Peptide cont. infusion |
| dose)/Agent | | | prior to ischemia and as | | for 180 min |
| | | | continuous infusion | | |
| | | | beginning at T + 40 min. | | |
| | | | and ongoing for 20 min. | | |
| | | | Agent administered as | | |
| | | | bolus dose at T + 60 min. | | |

Temperature and Hemodynamic Measurements.

Arterial blood pressure, left ventricular pressure, heart rate, surface electrocardiogram, and rectal temperature are continuously monitored throughout the protocol in all animals. Hemodynamic, heart rate, and temperature measurements are recorded at baseline, after initiation of peptide or placebo for peptide infusion, at 40 min of ischemia, immediately prior to and after the release of the coronary snares, and after 3 hours of reperfusion. The rate pressure product is calculated by multiplying the heart rate by the systolic blood pressure at all time points.

Analysis of Areas at Risk and Infarct Size.

At the completion of the protocol, the coronary snares are retightened; vascular clamps are used to occlude the aorta, pulmonary artery, and inferior vena cava; and the right atrium is incised. One milliliter per kilogram of Evans blue dye (Sigma, St. Louis, Mo.) is injected via the left atrium to delineate the ischemic myocardial risk area (AR).

All animals are euthanized via an injection of potassium chloride into the left atrium. Next, the heart is excised, and the LV is sectioned perpendicular to its long axis into six slices. The thickness of each slice is measured with a digital micrometer, and all slices are photographed. All slices are then incubated in 2% triphenyltetrazolium chloride (TTC) at 37° C. for 20 min and rephotographed. All photographs are imported into an image analysis program (Image Pro Plus, Media Cybernetics, Silver Spring, Md.). Myocardium unstained by Evans blue dye is determined to be the AR. Infarct area is determined by incubating the myocardium in TTC. TTC is a colorless dye, which is reduced to a brick-red colored precipitate in the presence of the coenzyme NADH. During reperfusion of previously ischemic myocardium, NADH is washed out of all necrotic myocytes. This results in a clear delineation of viable myocardium, which stains brick-red, and non-viable myocardium, which is visualized as an unstained, pale color. See, e.g., Leshnower et al., Am J Physiol Heart Circ Physiol 293: H1799-H1804, 2007, for exemplary images.

Computerized planimetry (Image Pro Plus, Media Cybernetics) is used to measure AR and infarct areas. AR is expressed as a percentage of the LV (AR/LV), and infarct size is expressed as a percentage of the AR (I/AR). AR and FAR are measured for the all slices, and a total AR and FAR for the entire LV is calculated.

Tissue Preparation.

The entire AR from LV slices are excised. A 1- to 2-mm transmural specimen is removed from the AR, snap frozen in liquid nitrogen, and stored at −80° C. The remainder of the AR is fixed for 24 hours in 10% formalin and subsequently embedded in paraffin.

In Situ Oligo Ligation Assay.

For the identification of apoptotic cells, an in situ oligo ligation (ISOL) assay (Intergen 7200; Intergen, Purchase, N.Y.) with a high specificity for staining the specific DNA fragmentation characteristic of apoptosis is selected. This assay utilizes T4 DNA ligase to bind synthetic biotinylated oligonucleotides to 3′-dT overhangs. Paraffin-embedded tissue is sectioned into 5-μm slices and deparaffinized by three changes of xylene, followed by three changes of absolute ethanol. Subsequently, endogenous peroxidase is quenched in 3% hydrogen peroxide in PBS. After washing the tissue sections, they are treated with 20 μg/mL proteinase K in PBS, washed again, and placed in an equilibration buffer. Next, a solution of T4 DNA ligase and oligonucleotides is applied to the slides and incubated overnight at 16° to 22° C. ApopTag detection of ligated oligonucleotides is accomplished by applying a streptavidin-peroxidase conjugate that is developed with diaminobenzidine. Finally, tissue sections are counterstained in hematoxylin.

Entire tissue sections are digitalized using a scanning microscope and analyzed using an image analysis software package (Image Pro Plus; MediaCybernetics, Silver Spring, Md.). ISOL-positive and ISOL-negative nuclei are counted in the AR. Results are expressed as an apoptic index, which is defined as the percentage of ISOL positive cells per total number of cells in the entire AR.

Transmurality Analysis.

Using advanced planimetry techniques (Image Pro Plus, MediaCybernetics), a transmural analysis is performed on the AR in the second slice from the apex to evaluate the spread of ischemic cell death within different regions of the myocardium. The second slice is selected because of its consistent appearance following ischemia and reperfusion from prior experiments. After basic planimetry is completed, the radius of the left ventricular wall is divided into three equivalent lengths at multiple points around the circumference, and individual arcs are created, which connected these radial points. Next, these arcs are connected circumferentially to form concentric ellipses, which divide the AR into three statistically equivalent areas (subendocardium, midmyocardium, and subepicardium; P=0.05). AR and FAR are measured.

Myocardial Fluorescence Spectroscopy.

Fluorescence spectroscopy of animal myocardium is conducted with a fluorometer. This fluorometer is a mobile optical-electrical apparatus that collects fluorescence signals of any type of tissue through a 3-mm-tip light guide catheter. The incident light is a broadband mercury arc lamp that can be filtered at two pairs of excitation/emission wavelengths by an air turbine filter wheel rotating at 50 Hz. Consequently, up to four signals can be multiplexed to a photodetector in order to make four-wavelength channel optical measurements of tissue metabolism. In this experiment two channels are used for excitation and the other two for emission signals. The light intensity that is incident on tissue at the fiber tip is 3 μW/mm². In cardiac fluorometry experiments, the excitation wavelengths of FAD and NADH are obtained by filtering the resonance lines of the mercury arc lamp at 436 nm and 366 nm by band-pass filters 440DF20 and 365HT25, respectively. The fluorescence intensities are then detected by a photomultiplier tube, converted to an electric voltage, digitized and displayed. Specific instrument specifications are kept the same for all the experiments.

The fluorometer catheter is placed on the epicardial surface in the center of the anticipated region of ischemia and continuous recording of the fluorescence signals for FAD and NADH signals is performed during 10 min of baseline, 60 min of infusion of saline or peptide, 30 min of ischemia, and 180 min of reperfusion. The redox ratio is calculated as FAD_{f}/FAD_{f}+NAD_{f}) every five minutes from the continuously recorded FAD and NAD. The redox ratio (RR) in each group are averaged and expressed as mean±standard error at five-minute time points for statistical analysis and ten-minute intervals for spectroscopic graphs.

Regional Blood Flow Measurements.

In test subjects, approximately fifteen million color-coded, 15.5 μm-diameter NuFlow Fluorescent microspheres (IMT Laboratories, Irvine, Calif.) are injected to measure the degree of ischemia during coronary occlusion and to study the effects of increasing ischemic time on microvascular integrity after reperfusion. Injections are made at baseline, after 30 min of ischemia, at the onset of reperfusion, and after 180 min of reperfusion. Reference blood samples are taken at all time points. At the end of the experiment, in a similar fashion to the transmural analysis described above, the AR from the second slice from the apex in each animal is isolated and circumferentially sectioned into three equivalent areas: subendocardium, midmyocardium, and subepicardium. The three different areas of myocardium and reference blood samples are analyzed using flow cytometry for microsphere content by IMT Laboratories. Regional perfusion is calculated using the following formula: Qₘ=(Cₘ×Qᵣ)/Cᵣ, where Qₘ is myocardial blood flow per gram ml min⁻¹ g⁻¹) of sample; Cₘ is microsphere count per gram of tissue in sample; Qᵣ is withdrawal rate of the reference blood sample ml/min); and Cᵣ is microsphere count in the reference blood sample. Regional blood flow (RBF) values are normalized and expressed as a percentage of baseline flow.

Analysis of Mitochondrial Disruption.

Three random tissue sections from the infarct region are embedded in EPON. One section is cut, stained, and analyzed, while the remaining two sections are archived for future analysis. Fifty mitochondria from all regions of the sample are assessed at a standardized magnification. The number of mitochondria with disrupted outer membranes are tallied and the percentage of disrupted mitochondria will be reported.

Transmission Electron Microscopy.

Myocardial punch biopsies are obtained from the AR from 2 animals from each of the control and peptide groups. Tissue is also obtained from 4 normal animals that are not subjected to the ischemia/reperfusion protocol. Biopsies are preserved in fixative (2.5% glutaraldehyde, 2.0% paraformaldehyde, 0.1 M sodium cacodylate [NaCaC]) for 24 hours at 4° C. After several washes in 0.1M NaCaC, samples are post-fixed with buffered 2% osmium tetroxide for 1 hour at 4° C. Subsequent washes in 0.1M NaCaC, water, and 2% aqueous uranyl acetate are used to destain samples. Tissue samples are dehydrated in serial washes of ethanol and propylene oxide, before a slow infiltration with EPON 812. Samples are cured at 70° C. for 48 hours and cut, stained, and imaged on a Jeol-10-10 transmission electron microscope (Jeol, Akishima, Japan). Random images are captured from each sample for comparative analysis. To assess the degree of mitochondrial disruption, five random images of mitochondria at 12,000 magnification per pig or sheep are captured from each specimen. Morphologic differences in mitochondria are assessed in the nuclear cap, a region surrounding the cell nucleus. The total number of mitochondria and the number of disrupted mitochondria are counted and averaged. The mean percentage of disrupted mitochondria is calculated and reported for each group.

The endpoints set forth in Table 9 will be measured using an appropriate technique known in the art, such as the exemplary techniques described in the preceding paragraphs.

TABLE 9
Experimental Endpoints.

| | Pre- | | | Short Term to | At End of | |
|---|---|---|---|---|---|---|
| Study Parameter to | Ischemic | Ischemic | Immediately | Longer Term | Reperfusion | At Post- |
| be Assessed | Period | Period | Post-Ischemia | Post-Ischemia | Period | Mortem |
| Cardiovascular | X | X | X | X | X | |
| Hemodynamics | | | | | | |
| ECG Waveforms and | X | X | X | X | X | |
| Intervals | | | | | | |
| Regional LV Wall | X | X | X | X | X | |
| Thickening | | | | | | |
| Mitochondrial | X | X | X | X | X | |
| Function | | | | | | |
| (REDOX State) | | | | | | |
| Mitochondrial | | | | | | X |
| Structure | | | | | | |
| Assessment of | | | | | | X |
| Apoptosis | | | | | | |
| LV Infarct Size, | | | | | | X |
| (AR/LV, IA/LV, | | | | | | |
| IA/AR) | | | | | | |

It is predicted that infarct size and apoptotic cell death in the peptide+cardiovascular agent-treated groups will be reduced compared to the control group. In some embodiments, it is predicted that the infarct size and apoptotic cell death in the peptide+cardiovascular agent-treated groups will be reduced at least 2%, at least 5%, at least 10%, at least 15%, at least 20%, at least 25%, or at least 50% compared to the control group. In some embodiments, non-responders will be excluded. It is also predicted that transmission electron microscopy will reveal a preservation of normal mitochondria morphology and a reduction in the percentage of disrupted mitochondria in the peptide+cardiovascular agent-treated group compared with the control group.

It is also predicted that the peptide+cardiovascular agent will influence mitochondrial function during both ischemia and reperfusion as indicated by the time course curves of the redox ratio (RR). The RR is calculated using intrinsic NAD and FAD fluorescence measurements is a sensitive index of mitochondrial metabolism. Since the fluorescence of NAD and FAD vary inversely with mitochondrial redox state the RR (FAD_{f}/FAD_{f}+NAD_{f})) has been found to correlate more strongly with mitochondrial function than either of the individual fluorescent measurements alone. In particular, it is predicted that when the peptide is given prior to ischemia, there is a reduced hypoxic-induced mitochondrial dysfunction indicated by a blunted drop in the RR during ischemia. Likewise, the RR is not expected to rise as quickly upon reperfusion the peptide+cardiovascular agent-treated groups as compared to the control groups.

These results will indicate that peptide and cardiovascular agent administration prevents the occurrence of symptoms of acute cardiac ischemia-reperfusion injury. Further, the combination therapy (peptide plus cardiovascular agent) will show improved results as compared to peptide treatment alone or cardiovascular agent alone. As such, the combination of cardiovascular agent and aromatic-cationic peptides are useful in methods at preventing and treating ischemia-reperfusion injury in mammalian subjects.

While the present example describes the use of the peptide D-Arg-2′6′-Dmt-Lys-Phe-NH₂ it is understood that other aromatic-cationic peptides disclosed herein could be tested and used with similar results.

### Example 3

### Effects of Combined Peptide and Cardiovascular Agent Treatment in Humans with Acute Myocardial Infarction Injury

This Example will determine whether the administration of D-Arg-2′6′-Dmt-Lys-Phe-NH₂ and a cardiovascular agent at the time of revascularization would limit the size of the infarct during acute myocardial infarction.

Study Group.

Men and women, 18 years of age or older, who present within 6 hours after the onset of chest pain, who have ST-segment elevation of more than 0.1 mV in two contiguous leads, and for whom the clinical decision is made to treat with percutaneous coronary intervention (PCI) are eligible for enrollment. Patients are eligible for the study whether they are undergoing primary PCI or rescue PCI. Occlusion of the culprit coronary artery (Thrombolysis in Myocardial Infarction [TIMI] flow grade 0) at the time of admission is also a criterion for inclusion.

Angiography and Revascularization.

Left ventricular and coronary angiography is performed with the use of standard techniques, just before revascularization. Revascularization is performed by PCI with the use of direct stenting. Alternative revascularization procedures include, but are not limited to, balloon angioplasty; insertion of a bypass graft; percutaneous transluminal coronary angioplasty; and directional coronary atherectomy.

Experimental Protocol.

After coronary angiography is performed but before the stent is implanted, patients who meet the enrollment criteria are randomly assigned to either the control group or the treatment group. Randomization is performed with the use of a computer-generated randomization sequence. Less than 10 min before direct stenting, the patients in the peptide group receive an intravenous bolus injection of D-Arg-2′6′-Dmt-Lys-Phe-NH₂ and a cardiovascular agent. The peptide is dissolved in normal saline (final concentration, 25 mg per milliliter) and is injected through a catheter that is positioned within an antecubital vein. Either separately or simultaneously, cardiovascular agent is injected through the catheter. Normal saline (0.9% NaCl) is used as a control. The patients in the control group receive an equivalent volume of normal saline.

Infarct Size.

The primary end point is the size of the infarct as assessed by measurements of cardiac biomarkers. Blood samples are obtained at admission and repeatedly over the next 3 days. The area under the curve (AUC) (expressed in arbitrary units) for creatine kinase and troponin I release (Beckman kit) is measured in each patient by computerized planimetry. The principal secondary end point is the size of the infarct as measured by the area of delayed hyperenhancement that is seen on cardiac magnetic resonance imaging (MRI), assessed on day 5 after infarction. For the late-enhancement analysis, 0.2 mmol of gadolinium-tetrazacyclododecanetetraacetic acid (DOTA) per kilogram is injected at a rate of 4 ml per second and is flushed with 15 ml of saline. Delayed hyperenhancement is evaluated 10 min after the injection of gadolinium-DOTA with the use of a three-dimensional inversion-recovery gradient-echo sequence. The images are analyzed in shortaxis slices covering the entire left ventricle.

Myocardial infarction is identified by delayed hyperenhancement within the myocardium, defined quantitatively by an intensity of the myocardial postcontrast signal that is more than 2 SD above that in a reference region of remote, noninfarcted myocardium within the same slice. For all slices, the absolute mass of the infracted area is calculated according to the following formula: infarct mass (in grams of tissue)=E (hyperenhanced area [in square centimeters])×slice thickness (in centimeters)×myocardial specific density (1.05 g per cubic centimeter).

Other End Points.

The whole-blood concentration of peptide and cardiovascular agent is measured immediately prior to PCI as well as at 1, 2, 4, 8 and 12 hours post PCI. Blood pressure and serum concentrations of creatinine and potassium are measured on admission and 24, 48, and 72 hours after PCI. Serum concentrations of bilirubin, γ-glutamyltransferase, and alkaline phosphatase, as well as white-cell counts, are measured on admission and 24 hours after PCI.

The cumulative incidence of major adverse events that occur within the first 48 hours after reperfusion are recorded, including death, heart failure, acute myocardial infarction, stroke, recurrent ischemia, the need for repeat revascularization, renal or hepatic insufficiency, vascular complications, and bleeding. The infarct-related adverse events are assessed, including heart failure and ventricular fibrillation. In addition, 3 months after acute myocardial infarction, cardiac events are recorded, and global left ventricular function is assessed by echocardiography (Vivid 7 systems; GE Vingmed).

It is predicted that administration of the peptide and cardiovascular agent at the time of reperfusion will be associated with a smaller infarct by some measures (endpoints) than that seen with placebo. As such, the combination of cardiovascular agent and aromatic-cationic peptides are useful in methods at preventing and treating ischemia-reperfusion injury in mammalian subjects.

While the present example describes the use of the peptide D-Arg-2′6′-Dmt-Lys-Phe-NH₂ it is understood that other aromatic-cationic peptides disclosed herein could be tested and used with similar results.

### REFERENCES

Leshnower B G, Kanemoto S, Matsubara M, Sakamoto H, Hinmon R, Gorman J H 3rd, Gorman R C. Cyclosporine preserves mitochondrial morphology after myocardial ischemia/reperfusion independent of calcineurin inhibition. Ann Thorac Surg., 2008 October, 86(4):1286-92.
Zhao L, Roche B M, Wessale J L, Kijtawornrat A, Lolly J L, Shemanski D, Hamlin R L. Chronic xanthine oxidase inhibition following myocardial infarction in rabbits: effects of early versus delayed treatment. Life Sci. 2008 Feb. 27; 82(9-10):495-502. Epub 2008 Jan. 24. PubMed PMID: 18215719.
Hamlin R L, Kijtawornrat A. Use of the rabbit with a failing heart to test for torsadogenicity. Pharmacol Ther. 2008 8 Aug., 119(2):179-85. Epub 2008 Apr. 20.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. Use of an aromatic-cationic peptide and a cardiovascular agent in the manufacture of a medicament for reducing infarct size and apoptotic cell death produced by AMI, wherein
the peptide is D-Arg-2′6′-Dmt-Lys-Phe-NH₂;
the cardiovascular agent is one or more of hyaluronidase, a corticosteroid, recombinant superoxide dismutase, prostacyclin, fluosol, magnesium, poloxamer 188, trimetazidine, eniporidine, cariporidine, a nitrate, anti-P selectin, an anti-CD18 antibody, adenosine, and glucose-insulin-potassium; and
wherein the medicament reduces infarct size and apoptotic cell death by at least 50% as compared to an untreated control.

2. The use of claim 1, wherein the cardiovascular agent is one or more of recombinant superoxide dismutase, magnesium, a nitrate, anti-P selectin, an anti-CD18 antibody, adenosine, and glucose-insulin-potassium.

3. The use of claim 1, wherein the cardiovascular agent is one or more of hyaluronidase, prostacyclin, fluosol, poloxamer 188, trimetazidine, eniporidine, and cariporidine.

4. The use of claim 1, wherein the cardiovascular agent is one or more corticosteroids selected from the group consisting of hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate.

5. Use of an aromatic-cationic peptide and a cardiovascular agent in the manufacture of a medicament for reducing infarct size and apoptotic cell death produced by AMI, wherein
the peptide is D-Arg-2′6′-Dmt-Lys-Phe-NH₂;
the cardiovascular agent is one or more of an anti-arrhythmia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a thromboxane receptor antagonist, a radical scavenger, an anti-platelet drug, a β-adrenaline receptor blocking drug, an α-receptor blocking drug, a sympathetic nerve inhibitor, a digitalis formulation, an inotrope, and an antihyperlipidemic drug; and
wherein the medicament reduces infarct size and apoptotic cell death by at least 50% as compared to an untreated control.

6. Use of claim 5, wherein the cardiovascular agent is one or more anti-arrhythmia agents selected from the group consisting of lidocaine, lignocaine moricizine, mexiletine, tocainide, procainamide, encainide, flecanide, tocainide, phenytoin, propafenone, quinidine, disopyramide, flecainide, propranolol, esmolol, amiodarone, artilide, bretylium, clofilium, isobutilide, sotalol, azimilide, dofetilide, dronedarone, ersentilide, ibutilide, tedisamil, trecetilide, verapamil, diltaizem, digitalis, adenosine, nickel chloride, and magnesium ions.

7. Use of claim 5, wherein the cardiovascular agent is one or more vasodilators selected from the group consisting of bencyclane, cinnarizine, citicoline, cyclandelate, cyclonicate, ebumamonine, hydralazine phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostaglandin E1, prostaglandin I2, an endothelin receptor blocking drug, diltiazem, nicorandil, and nitroglycerin.

8. Use of claim 5, wherein the cardiovascular agent is one or more anti-anginal agents selected from the group consisting of nitrates, isosorbide nitrate, glyceryl trinitrate, and pentaerythritol tetranitrate.

9. Use of claim 5, wherein the cardiovascular agent is one or more cardioglycosides selected from the group consisting of digoxin and digitoxin.

10. Use of claim 5, wherein the cardiovascular agent is one or more diuretics selected from the group consisting of thiazide diuretics, loop diuretics, K⁻ sparing diuretics, osmotic diuretics, nonthiazide diuretics, and acetazolamide.

11. Use of claim 5, wherein the cardiovascular agent is one or more sedatives selected from the group consisting of nitrazepam, flurazepam and diazepam.

12. Use of claim 5, wherein the cardiovascular agent is one or more ACE inhibitors selected from the group consisting of captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril.

13. Use of claim 5, wherein the cardiovascular agent is one or more angiotensin II antagonists selected from the group consisting of losartan, candesartan, valsartan, eprosartan, and irbesartan.

14. Use of claim 5, wherein the cardiovascular agent is one or more thrombolytic agents selected from the group consisting of tissue-type plasminogen activators, nasaruplase, streptokinase, urokinase, prourokinase, anisoylated plasminogen streptokinase activator complex, aspirin, heparin, warfarin that inhibits Vit K-dependent factors, low molecular weight heparins that inhibit factors X and II, thrombin inhibitors, inhibitors of platelet GP IIbIIIa receptors, inhibitors of tissue factor (TF), inhibitors of human von Willebrand factor, reptilase, TNK-t-PA, staphylokinase, and animal salivary gland plasminogen activators.

15. Use of claim 5, wherein the cardiovascular agent is one or more calcium channel blockers selected from the group consisting of aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline.

16. Use of claim 5, wherein the cardiovascular agent is one or more thromboxane receptor antagonists selected from the group consisting of ifetroban, prostacyclin mimetics, and phosphodiesterase inhibitors.

17. Use of claim 5, wherein the cardiovascular agent is one or more antiplatelet drugs selected from the group consisting of ticlopidine hydrochloride, dipyridamole, cilostazol, ethyl icosapentate, sarpogrelate hydrochloride, dilazep hydrochloride, trapidil, a nonsteroidal antiinflammatory agent, beraprostsodium, iloprost, and indobufene.

18. Use of claim 5, wherein the cardiovascular agent is one or more β-adrenaline receptor blocking drugs selected from the group consisting of propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol.

19. Use of claim 5, wherein the cardiovascular agent is one or more α-receptor blocking drugs selected from the group consisting of amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine.

20. Use of claim 5, wherein the cardiovascular agent is one or more sympathetic nerve inhibitors selected from the group consisting of clonidine, guanfacine, guanabenz, methyldopa, reserpine, hydralazine, todralazine, budralazine, and cadralazine.

21. Use of claim 5, wherein the cardiovascular agent is one or more digitalis formulations selected from the group consisting of digitoxin, digoxin, methyldigoxin, deslanoside, vesnarinone, lanatoside C, and proscillaridin.
